(19) United States
(12) Patent Application Publication
(10) Pub. No.: US 2017/0115275 A1
(43) Pub. Date: Apr. 27, 2017

(54) ENGINEERED SUBSTRATES FOR HIGH-THROUGHPUT GENERATION OF 3D MODELS OF TUMOR DORMANCY, RELAPSE AND MICROMETASTASES FOR PHENOTYPE SPECIFIC DRUG DISCOVERY AND DEVELOPMENT

(71) Applicants: Kaushal Rege, Chandler, AZ (US); Taraka Sai Pavan Grandhi, Tempe, AZ (US); Thrimoorthy Potta, Phoenix, AZ (US)

(72) Inventors: Kaushal Rege, Chandler, AZ (US); Taraka Sai Pavan Grandhi, Tempe, AZ (US); Thrimoorthy Potta, Phoenix, AZ (US)

(73) Assignee: Arizona Board of Regents on behalf of Arizona State University, Scottsdale, AZ (US)

(21) Appl. No.: 15/332,928

(22) Filed: Oct. 24, 2016

Related U.S. Application Data
(60) Provisional application No. 62/245,865, filed on Oct. 23, 2015.

(51) Int. Cl. G01N 33/50; C12N 5/09; C08J 3/075; C12N 5/00

(52) U.S. Cl. CPC G01N 33/5008; C12N 5/0062; C12N 5/0693; C08J 3/075; C12N 2533/30; C12N 2537/10; C08J 2363/00

(57) ABSTRACT

Methods to form a novel aminoglycoside based hydrogel for high-throughput generation of 3D dormant, relapsed and micrometastatic tumor microenvironments are disclosed. In addition, methods of screening agents against tumor cells grown in the 3D environments disclosed herein that include, for example, screening of lead drugs and therapies for an effect on dormant, relapsed and/or micrometastatic tumor cells.

### CROSS-REFERENCE TO RELATED APPLICATIONS

This application claims priority to U.S. Provisional Patent Application No. 62/245,865, filed on Oct. 23, 2015, which is incorporated herein by reference as if set forth in its entirety.

### STATEMENT REGARDING FEDERALLY SPONSORED RESEARCH

This invention was made with government support under RO1GM093229 awarded by the National Institutes of Health. The government has certain rights in the invention.

### FIELD OF INVENTION

This disclosure generally relates to a novel aminiglycoside based hydrogel for high-throughput generation of 3D dormant, relapsed and micrometastatic tumor microenvironemnts.

### BACKGROUND

Tumor resistance to drugs severely limits the success of modern chemotherapy in eliminating cancer. Upon exposure to chemotherapy, sensitive cancer cells are eliminated, while resistant and dormant cells that do not respond to treatment survive. Ultimately, these dormant and resistant cells repopulate, causing a relapse of the disease at the primary location, as well as at distant metastatic sites. Dormant cells can exist either as minimal residual disease in which, cells are present at the site of primary tumor after surgical resection, or as distant disseminated cells in metastatic sites such as bone, liver and lymph nodes. Often in cases of metastasis, tumor cells initially undergo prolonged periods of dormancy, which are followed by relapse. Engineered high-throughput systems of tumor dormancy and resistance are desired for large scale screening of lead drugs and therapies, aiding cancer drug discovery and delivery. Cancer cell models that can capture tumor complexity and serve as high-throughput systems are needed.

### SUMMARY

Embodiments of Applicant disclosure describe a cross-linked hydrogel, which comprises an aminoglycoside and a polymeric compound. In certain embodiments, the aminoglycoside is (2S)-4-amino-N-[(1R,2S,3S,4R,5S)-5-amino-2-[(2S,3R,4S,5S,6R)-4-amino-3,5-dihydroxy-6-(hydroxymethyl)oxan-2-yl]oxy-4-[(2R,3R,4S,5S,6R)-6-(aminomethyl)-3,4,5-trihydroxyoxan-2-yl]oxy-3-hydroxycyclohexyl]-2-hydroxybutanamide, and/or salt or hydrate thereof; and the polymeric compound is poly (ethylene glycol) diglycidyl ether.

In certain embodiments, the cross-linked hydrogel comprises various degrees of mechanical stiffness of about 7 kilopascals (KPa) to about 100 KPa. In other embodiments, the cross-linked hydrogel comprises a non-adhesive surface.

Further, methods to generate a microenvironment (3DTM) using said cross-linked hydrogel are disclosed. In certain embodiments, the method comprises overlaying a first plurality of cancer cells and culturing said cancer cells under conditions and for a duration sufficient to form a spheroidal 3DTM. In other embodiments, the overlaying step further comprises a second plurality of stromal cells.

In certain embodiments, the plurality of cancer cells is selected from the group consisting of T24 bladder cancer cells, PC3 prostate cancer cells, PC3-eGFP prostate cancer cells, and MDA-MB-231 breast cancer cells. In other embodiments, the second plurality of stromal cells is selected from the group consisting of NIH3T3 murine fibroblasts, BJ-5ta human foreskin fibroblasts, and WPMY-1 human prostate stromal cells.

In addition, in certain embodiments, the size of the spheroidal 3DTM is dependent on the seeding density of the plurality of cancer cells. Moreover, in certain embodiments, the spheroidal 3DTM comprises greater than 80% cells that are arrested in a G0/G1 phase of the cell cycle. In other embodiments, the spheroidal 3DTM comprises greater than 95% cells that are arrested in a G0/G1 phase of the cell cycle.

A method of screening an agent against tumor cells cultured in the spheroidal 3DTM is also disclosed. In certain embodiments, the screening method comprises contacting said tumor cells with said agent and determining an effect on said cells. Further, said contacting step comprises an agent that induces ER stress; and further comprises contacting said cells with a second agent that modulates intracellular calcium levels. In certain embodiments, said agent is a drug, antibody, biologic, or a combination thereof.

### BRIEF DESCRIPTION OF DRAWINGS

FIG. 1A is a schematic of Amikagel formation (i) Amikacin hydrate and poly (ethylene glycol) diglycidyl ether (PEGDE) were mixed in nanopure water at room temperature. (ii). Characteristic (A) sol to (B) gel transition of Amikagel at 40° C. temperature after 7.5 h of incubation. (C) Amikagel held between fingers;

FIG. 1B shows the time required for formation of Amikagels AM1, AM2 and AM3 as a function of temperature;

FIGS. 1C and 1D are characterization of Amikagels using electron microscopy and swelling studies. Scanning Electron Microscopy (SEM) images of (A) different Amikagel surfaces, and (B) fractured Amikagels. FIG. 1D shows swelling ratios of Amikagels after 48 h incubation at room temperature in Nanopure water. Lower crosslinking ratios led to higher swelling of the Amikagels, likely due to porous crosslinks, which could absorb water to swell up.

FIG. 2 shows (A) High-throughput generation of 3D tumor microenvironments (3DTMs) on AM3 Amikagel; the 3DTM (0.8-1 mm diameter) can be visualized using the naked eye (arrow). (B) Phase-contrast and fluorescence (Live-Dead®) images of breast, and bladder cancer 3DTMs. Scale=100 μm. (C) Representative phase-contrast image of high-throughput generation of T24 3DTMs in 24 wells coated with Amikagels; ‘one well-one 3DTM’ formation, with near-uniform sized 3DTMs, can be seen 7 days of initial seeding. Scale=100 μm;

FIG. 3A are Scanning Electron Microscopy (SEM) images of (i-v) NIH3T3-T24 coculture 3DTMs. Images ii-v show NIH3T3-T24 3DTM at higher magnifications, with most cells showing a rounded morphology. NIH3T3-T24 3DTMs were covered with fibrous material indicative of extracellular matrix (ECM) formation (red arrows);

FIG. 3B is Hematoxylin and Eosin (H&E) staining of 3DTMs NIH3T3-T24 3DTM. Scale=100 μm and insets (i—central necrotic core, ii—middle densely packed cells and iii—outer loosely packed cells) Scale=20 μm. Yellow pointers indicate condensed nuclei indicative of pyknosis;

FIGS. 4A-4C show Dormancy relapse of T24 cells: Dormant T24 3DTMs were transferred from AM3 Amikagels to AM1 Amikagel and visualized for changes in spheroid disassembly. Representative images are shown. Phase contrast image of the transferred 3DTM after (A). Day 0 after transfer, (B). Day 1 of transfer, (C). Day 15 after transfer. Following transfer of dormant T24 3DTMs from AM3 amikagels, cell shedding on AM1 Amikagels resulted in the formation of microcolonies, 70-100 μm diameter, within 15 days following transfer. Representative image is shown in D Scales=100 μm in all cases;

FIGS. 4D-4F illustrate Escape from dormancy is accompanied by changes in cell cycle. (D). Cell cycle distribution of the remnant dormant ‘mother’ T24 3DTM indicated near-complete arrest in the G0/G1 phase. (E). Cell cycle profiles of cells that escape the ‘mother 3DTM’, spread and colonize different regions on AM1 Amikagel, (F). Quantitative analysis of cell cycle—In all cases, 3DTMs were formed on AM3 Amikagels for 7 days transferred to AM1 Amikagel. Cell cycle analyses were carried out on the two populations (‘mother 3DTM and escaped cells) 7 days following transfer. Statistically significant difference was found between the G2/M phases of cell cycle distributions of the escaped cells and the dormant mother 3DTM, indicating an actively proliferating population in the shed cells (*p=0.004; Student's t-test);

FIG. 5A shows cell death (%) after exposure of dormant T24 to docetaxel for 96 hours after 3DTM formation estimated by flow cytometry after treating with Live-Dead® stain (Calcein AM-EthD-1) (squares). Minimal cell death of dormant cancer cells against docetaxel was measured by using XTT assay showed similar result. Cell death (%) after exposure of 5000 T24 cells to docetaxel for 96 hours after 2D cell culture measured by using MTT assay (circles). LC50=10 μM. Dormant T24 3DTMs showed very high resistance to conventional chemotherapies;

FIG. 5B illustrates total protein content in dormant cells was found to be significantly higher than actively dividing cells (n=3, p<0.05, 1.3 fold higher, Student's t-test);

FIG. 5C demonstrates that ER stress inducers (SERCA inhibitor thapsigargin) that cause protein misfolding in concert with ER protein accumulators could amplify unfolded protein response to cause cell death. Autophagic and proteasome pathways were targeted using wortmannin and bortezomib drugs respectively;

FIG. 5D shows combination of ER stress inducer thapsigargin coupled with proteasome inhibitor bortezomib led to synergistic increase in cell death in dormant cancer cell systems (96 hour treatment). (Combination index (CIBortezomib+Thapsigargin=0.1±0.025, indicating very strong synergy between bortezomib and thapsigargin). Wortmannin and thapsigargin did not induce synergistic cell death. (i) Single agent thapsigargin, bortezomib and wortmannin treatment on dormant T24 spheroids is shown. (ii) 0.5 μM treatment with bortezomib and wortmannin caused similar amounts of cell death (fewer than 10% cell death). (iii) Bortezomib with thapsigargin was much better than wortmannin and thapsigargin in inducing cell death that was reversed using pancaspase inhibitor zVAD-fmk. Reversal of cell death after pan-caspase inhibition indicated an apoptotic cell death induction during bortezomib-thapsigargin treatment;

FIG. 5E shows dormant T24 3DTMs were treated with bortezomib (0.5 μM), thapsigargin (0.5 μM), high concentration calcimycin (5 μM) and calcium (5 mM) and their combinations for 24 to 96 hours (sequence of bars). While bortezomib and thapsigargin induced only 2% death in 24 hours, their combination with calcium and calcimycin (5 μM) induced significantly higher death (p<0.001) (˜60%);

FIG. 5F shows calcimycin dose response on dormant 3DTMs showed an LC50 of around 10 μM after a treatment for 24 hours that was not reversible via pan-caspase inhibition indicating a necrotic cell death induction rather than apoptosis (shown in supporting information). Calcimycin was significantly more toxic compared to bortezomib and thapsigargin in 24 hours. Supplementation of 5 mM calcium with calcimycin (checker bars) increased the cell death impact of calcimycin;

FIG. 5G shows dormant T24 3DTMs were treated with bortezomib (0.5 μM), thapsigargin (0.5 μM), low concentration calcimycin (0.5 μM) and calcium (5 mM) and their combinations for 24 to 96 hours (sequence of bars). While bortezomib and thapsigargin induced only 11% death in 48 hours, their combination with calcium and calcimycin (0.5 μM) induced significantly higher death (p<0.001) (˜65%). The acceleration was reversible upon addition of pan-caspase inhibitor (5 μM zVAD-fink) indicating an apoptotic cell death acceleration;

FIG. 5H illustrates that 48 hour data point of the treatment is—bortezomib (0.5 μM), thapsigargin (0.5 μM), low concentration calcimycin (0.5 μM) and calcium (5 mM). Acceleration of cell death (˜65%, (p<0.001)) after addition of calcimycin (0.5 μM) and calcium with bortezomib and thapsigargin is shown. The acceleration was reversible upon addition of pan-caspase inhibitor (5 μM zVAD-fmk) indicating an apoptotic cell death acceleration (One way ANOVA, multiple comparisons);

FIG. 5I illustrates calcium loaded liposomes (175 μM) were seen to maintain the acceleration in cell death in combination with bortezomib (0.5 μM) and thapsigargin (0.5 μM), as observed previously by calcimycin (48 and 72 hour data points are shown). Empty DPPC liposomes were also seen to cause a certain increase in cell death;

FIG. 6A shows CHOP expression after 24 hour treatment with calcium (5 mM), calcimycin (5 μM; higher concentration), bortezomib and thapsigargin (0.5 μM each);

FIG. 6B shows mitochondrial depolarization imaged using JC-1 dye after 24 hour treatment with calcium (5 mM), calcimycin (5 μM; higher concentration), bortezomib and thapsigargin (0.5 μM each) (Green=Depolarized mitochondria, Red=Normal mitochondria, Lower Red/Green ratio=higher mitochondrial depolarization);

FIG. 6C shows mitochondrial depolarization imaged using JC-1 dye after 24 hour treatment with calcium (5 mM), calcimycin (0.5 μM; lower concentration), bortezomib and thapsigargin (0.5 μM-each) (Green=Depolarized mitochondria, Red=Normal mitochondria, Lower Red/Green ratio=higher mitochondrial depolarization);

FIG. 6D illustrates cytoplasmic calcium supplementation in presence of acute ER stress could accelerate mitochondrial depolarization and apoptotic cell death;

FIGS. 7A-7E show effect of chemotherapeutic drug (docetaxel) treatment on escape from dormancy of T24 bladder cancer 3DTMs. (A). Experimental sequence. (B). Representative image of dormant T24 3DTM grown on AM3 Amikagel and transferred to AM1 Amikagel; this 3DTM was not treated with docetaxel. Image taken after 48 hours of transfer of dormant T24 3DTM to AM1 gel showed abundant cell escape out of the mother spheroid. (C). Representative image of dormant T24 3DTM formed and subsequently treated with 100 μM docetaxel on AM3 Amikagel. The pretreated 3DTM was then transferred to AM1 Amikagel. Image taken after 48 hours of transfer of the docetaxel pre-treated dormant T24 3DTM to AM1 gel. As seen in the picture, significantly lesser number of cells escaped the mother spheroid after treatment with docetaxel. Scale=100 μm in all cases. Docetaxel further inhibited microcolony formation: Microcolony formation by (D) untreated and (E) 100 μM docetaxel-treated T24 3DTMs after 15 days of transfer to AM1 Amikagel. Docetaxel treatment significantly reduced cell escape and thus micro-colony formation. Scale=100 μm in all cases;

FIGS. 8A-8C illustrate surface adhesivity of (A) T24 bladder cancer cells, and (B) WPMY-1 prostate stromal cells on AM1, AM2 and AM3 Amikagels following 24 hours of seeding is shown. Elongated cellular appendages can be on AM1 and AM2 Amikagels (arrow heads), but are absent on cells cultured on AM3 Amikagel, indicating poor cell adhesion to AM3 Amikagel. Cells come together on AM3 cells, ultimately resulting in the formation of 3DTMs. Scale: 100 μm. (C) Qualitative measurement of amikagel adhesivity compared to 2D tissue culture plastic indicated ˜99.5% lower adhesivity for AM3 gel respectively;

FIGS. 9A-9B show control of 3DTM size by using different cell seeding densities/well in a 96-well plate coated with AM3 Amikagel. Scale=100 μm. (B). Kinetics of 3DTM formation: T24 cells, NIH3T3-T24 and NIH3T3-eGFP-PC3 co-culture 3DTMs. Cells assembled into a pre-spheroidal ‘sheet’, which subsequently rolled upon itself and compacted, resulting in the formation of 3DTMs. Representative images are shown in this figure. Scale=100 pin;

FIGS. 10A-10D show Cell cycle analysis of 3DTMs: (a) 2D vs. (b) 3DTM for T24 cells and (c) 2D vs. (d) 3DTM for UMUC3 cells ** indicates p<0.0001 for G0/G1 population compared for 2D and 3DTM of T24 and UMUC3 cells, respectively. (n=3, independent experiments). Statistical significance determined using Student's t-test;

FIGS. 11A & 11B show treatment of T24 3DTMs with mitoxantrone and docetaxel (A). Live/Dead® staining of disassembled T24 3DTM after 96 hours of exposure with different doses of mitoxantrone. Increasing the concentration of mitoxantrone did not have significant impact on the cell viability of T24 cells in the 3DTMs. Scale=100 μm. (B). Live/Dead® staining of disassembled T24 3DTM after 96 hours of exposure to different doses of docetaxel. Increasing the concentration of Docetaxel did not have any impact on the cell viability of T24 cells in the 3DTM. Scale=100 μm.

### DETAILED DESCRIPTION

The disclosure herein relates to the generation of 3D cancer cell models that capture tumor complexity, reduced drug and metabolite transport, drug and radiation resistance, and hypoxia generated with an aminiglycoside based hydrogel with different chemical and/or mechanical characteristics. Particularly, the generation of three-dimensional tumor models (3DTMs) with the aminiglycoside based hydrogel demonstrate cellular dormancy and resistance; possesses high mechanical stiffness in concert with non-adhesive surface chemistry; and facilitate rapid drug screening in the context of tumor resistance and dormancy.

This disclosure is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

As described herein, a gel is a solid jelly-like material that can have properties ranging from soft and weak to hard and tough. Gels are defined as a substantially dilute cross-linked system, which exhibits no flow when in the steady-state. By weight, gels are mostly liquid, yet they behave like solids due to a three-dimensional cross-linked network within the liquid. It is the crosslinking within the fluid that gives a gel its structure (hardness) and contributes to the adhesive stick (tack). In this way gels are a dispersion of molecules of a liquid within a solid in which the solid is the continuous phase and the liquid is the discontinuous phase. Gels consist of a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids. Furthermore, a hydrogel is a network of polymer chains that are hydrophilic, sometimes found as a colloidal gel in which water is the dispersion medium. Hydrogels are highly absorbent (they can contain over 90% water) natural or synthetic polymeric networks. Hydrogels also possess a degree of flexibility very similar to natural tissue, due to their significant water content.

As described herein, molecular mass or molecular weight is the mass of a molecule. It is calculated as the sum of the atomic mass of each constituent atom multiplied by the number of atoms of that element in the molecular formula. Both atomic and molecular masses are usually obtained relative to the mass of the isotope 12C (carbon 12), which by definition is equal to 12. A more proper term would be “relative molecular mass”. Relative atomic and molecular mass values are dimensionless but are given the “unit” Dalton (formerly atomic mass unit) to indicate that the number is equal to the mass of one molecule divided by 1/12 of the mass of one atom of 12C. The mass of 1 mole of substance is designated as molar mass. By definition, it has the unit gram. The atomic weight of carbon is given as 12.011, not 12. This is because naturally occurring carbon is a mixture of the isotopes 12C, 13C and 14C which have relative atomic masses of 12, 13 and 14 respectively. Moreover, the proportion of the isotopes varies between samples, so 12.011 is an average value. The molecular mass of small to medium size molecules, measured by mass spectrometry, determines stoichiometry. For large molecules such as proteins, methods based on viscosity and light-scattering can be used to determine molecular mass when crystallographic data are not available.

As described herein, storage (G′) and loss (G″) moduli were experimentally determined as a function of applied frequency and absolute shear modulus (|G*|). The storage modulus (G′) gives information about material elastic properties and its mechanical stiffness, while loss modulus (G″) provides information about the viscous/liquid properties of the material. Absolute shear modulus representing the stiffness of the hydrogel was calculated as |G*|=(G′²+G″²)^{0.5}.

As described herein, the number average molar mass is a way of determining the molecular mass of a polymer. Polymer molecules, even ones of the same type, come in different sizes (chain lengths, for linear polymers), so the average molecular mass will depend on the method of averaging. The number average molecular mass is the ordinary arithmetic mean or average of the molecular masses of the individual macromolecules. It is determined by measuring the molecular mass of n polymer molecules, summing the masses, and dividing by n. The number average molecular mass is calculated by

$${\tilde{M}}_{n} = \frac{{\sum }_{i} {N}_{i } {M}_{i}}{{\sum }_{i} {N}_{i }}$$

The mass average molar mass (often loosely termed weight average molar mass) is another way of describing the molar mass of a polymer. Some properties are dependent on molecular size, so a larger molecule will have a larger contribution than a smaller molecule. The mass average molar mass is calculated by

$${\tilde{M}}_{w} = \frac{{\sum }_{i} {N}_{i } {M}_{i}^{2}}{{\sum }_{i} {N}_{i } {M}_{i}}$$

where is the number of molecules of molecular mass

As described herein, the term “salt” refers to any ionic form of a compound and one or more counter-ionic species (cations and/or anions). The term “salt” additionally includes zwitterionic compounds (i.e., a molecule containing one more cationic and anionic species, e.g., zwitterionic amino acids). Counter ions present in a salt can include any cationic, anionic, or zwitterionic species. Examples of anions include, but are not limited to: chloride, bromide, iodide, nitrate, sulfate, bisulfate, sulfite, bisulfite, phosphate, acid phosphate, perchlorate, chlorate, chlorite, hypochlorite, periodate, iodate, iodite, hypoiodite, carbonate, bicarbonate, isonicotinate, acetate, trichloroacetate, trifluoroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluormethansulfonate, ethanesulfonate, benzensulfonate, p-toluenesulfonate, p-trifluoromethylbenzenesulfonate, hydroxide, aluminates and borates. Examples of cations include, but are not limited to: monovalent alkali, metal cations, such as lithium, sodium, potassium, and cesium, and divalent alkaline earth metals, such as beryllium, magnesium, calcium, strontium, and barium. Also covered by this term are transition metal cations, such as gold, silver, copper and zinc, as well as non-metal cations, such as ammonium salts.

In certain embodiments, a cross-linked hydrogel comprises an aminoglycoside, wherein the aminoglycoside is selected from the group consisting of 2-[(1R,2R,3S,4R,5R,6S)-3-(diaminomethylideneamino)-4-[(2R,3R,4R,5 S)-3-[(2S,3S,4S,5R,6S)-4,5-dihydroxy-6-(hydroxymethyl)-3-(methylamino)oxan-2-yl]oxy-4-formyl-4-hydroxy-5-methyloxolan-2-yl]oxy-2,5,6-trihydroxycyclohexyl]guanidine (herein after Streptomycin), (2R,3S,4R,5R,6R)-5-amino-2-(aminomethyl)-6-[(1R,2R,3S,4R,6S)-4,6-diamino-2-[(2S,3R,4S,5R)-4-[(2R,3R,4R,5S,6S)-3-amino-6-(aminomethyl)-4,5-dihydroxyoxan-2-yl]oxy-3-hydroxy-5-(hydroxymethyl)oxolan-2-yl]oxy-3-hydroxycyclohexyl]oxyoxane-3,4-diol (herein after neomycin or neomycin b), (2S,3S,4R,5R,6R)-5-amino-2-(aminomethyl)-6-[(2R,3S,4R,5 S)-5-[(1R,2R,3S,5R,6S)-3,5-diamino-2-[(2S,3R,4R,5S,6R)-3-amino-4,5-dihydroxy-6-(hydroxymethyl)oxan-2-yl]oxy-6-hydroxycyclohexyl]oxy-4-hydroxy-2-(hydroxymethyl)oxolan-3-yl]oxyoxane-3,4-diol (herein after paromomycin), (2R,3S,4R,5R,6R)-5-amino-2-(aminomethyl)-6-[(1R,2R,3S,4R,6S)-4,6-diamino-2-[(2S,3R,4S,5R)-3,4-dihydroxy-5-(hydroxymethyl)oxolan-2-yl]oxy-3-hydroxycyclohexyl]oxyoxane-3,4-diol (herein after ribostamycin), (2R,3S,4S,5R,6R)-2-(aminomethyl)-6-[(1R,2R,3S,4R,6S)-4,6-diamino-3-[(2S,3R4S,5S,6R)-4-amino-3,5-dihydroxy-6-(hydroxymethyl)oxan-2-yl]oxy-2-hydroxycyclohexyl]oxyoxane-3,4,5-triol (herein after kanamycin), (2S)-4-amino-N-[(1R,2S,3S,4R,5 S)-5-amino-2-[(2S,3R,4S,5S,6R)-4-amino-3,5-dihydroxy-6-(hydroxymethyl)oxan-2-yl]oxy-4-[(2R,3R,4S,5S,6R)-6-(aminomethyl)-3,4,5-trihydroxyoxan-2-yl]oxy-3-hydroxycyclohexyl]-2-hydroxybutanamide (herein after amikacin), (2S)-4-amino-N-[(1R,2S,3S,4R,5 S)-5-amino-4-[(2R,3R,6S)-3-amino-6-(aminomethyl)oxan-2-yl]oxy-2-[(2S,3R,4S,5S,6R)-4-amino-3,5-dihydroxy-6-(hydroxymethyl)oxan-2-yl]oxy-3-hydroxycyclohexyl]-2-hydroxybutanamide (herein after arbekacin), (2R,3S,4R,5R,6R)-5-amino-2-(aminomethyl)-6-[(1S,2R,3R,4S,6R)-4,6-diamino-3-[(2S,3R,4S,5S,6R)-4-amino-3,5-dihydroxy-6-(hydroxymethyl)oxan-2-yl]oxy-2-hydroxycyclohexyl]oxyoxane-3,4-diol (herein after as bekanamycin), (2S,3R,4S,5S,6R)-4-amino-2-[(1S,2S,3R,4S,6R)-4,6-diamino-3-[(2R,3R,6S)-3-amino-6-(aminomethyl)oxan-2-yl]oxy-2-hydroxycyclohexyl]oxy-6-(hydroxymethyl)oxane-3,5-diol (herein after as dibekacin), (2S,3R,4S,5S,6R)-4-amino-2-[(1S,2S,3R,4S,6R)-4,6-diamino-3-[(2R,3R,5S,6R)-3-amino-6-(aminomethyl)-5-hydroxyoxan-2-yl]oxy-2-hydroxycyclohexyl]oxy-6-(hydroxymethyl)oxane-3,5-diol (herein after as tobramycin), (1R,3S,5R,8R,10R,11S,12S,13R,14S)-8,12,14-hydroxy-5-methyl-11,13-bis(methylamino)-2,4,9-trioxatricyclo[8.4.0.0³'S]tetradecan-7-one (herein after as spectinomycin), 4-[3-amino-2,6-dihydroxy-5-(methylamino)cyclohexyl]oxy-6′-(1-amino-2-hydroxyethyl)-6-(hydroxymethyl)spiro[4,6,7,7a-tetrahydro-3aH-[1,3]dioxolo[4,5-c]pyran-2,2′-oxane]-3′,4′,5′,7-tetrol (herein after as hygromycin b), 2-[4,6-diamino-3-[3-amino-6-[1-(methylamino)ethyl]oxan-2-yl]oxy-2-hydroxycyclohexyl]oxy-5-methyl-4-(methylamino)oxane-3,5-diol (herein after as gentamicin), (2R,3R,4R,5R)-2-[(1S,2S,3R,4S,6R)-4-amino-3-[[(2S,3R)-3-amino-6-(aminomethyl)-3,4-dihydro-2H-pyran-2-yl]oxy]-6-(ethylamino)-2-hydroxycyclohexyl]oxy-5-methyl-4-(methylamino)oxane-3,5-diol (netilmicin), (2R,3R,4R,5R)-2-[(1S,2S,3R,4S,6R)-4,6-diamino-3-[[(2S,3R)-3-amino-6-(aminomethyl)-3,4-dihydro-2H-pyran-2-yl]oxy]-2-hydroxycyclohexyl]oxy-5-methyl-4-(methylamino)oxane-3,5-diol (herein after as sisomicin), (2S)-3-amino-N-[(1R,2S,3S,4R,5 S)-5-amino-4-[(2R,3R,4S,5S,6R)-6-(aminomethyl)-3,4,5-trihydroxyoxan-2-yl]oxy-2-[(2R,3R,4R,5R)-3,5-dihydroxy-5-methyl-4-(methylamino)oxan-2-yl]oxy-3-hydroxycyclohexyl]-2-hydroxypropanamide (herein after as isepamicin), (2R,3R,4R,5R)-2-[(1S,2S,3R,4S,6R)-4,6-diamino-3-[[(2S,3R)-3-amino-6-[(1 S)-1-aminoethyl]-3,4-dihydro-2H-pyran-2-yl]oxy]-2-hydroxycyclohexyl]oxy-5-methyl-4-(methylamino)oxane-3,5-diol (herein after as verdamicin),2-amino-N-[(1S,2R,3R,4S,5S,6R)-4-amino-3-[(2R,3R,6S)-3-amino-6-[(1 S)-1-aminoethyl]oxan-2-yl]oxy-2,5-dihydroxy-6-methoxycyclohexyl]-N-methylacetamide (herein after as astromicin), (2R,3R,4S,5S,6S)-2-[[(2R,3S,4R,4aR,6S,7R,8aS)-7-amino-6-[(1R,2R,3S,4R,6S)-4,6-diamino-2,3-dihydroxycyclohexyl]oxy-4-hydroxy-3-(methylamino)-2,3,4,4a,6,7,8,8a-octahydropyrano[3,2-b]pyran-2-yl]oxy]-5-amino-6-(hydroxymethyl)oxane-3,4-diol; sulfuric acid (herein after as apramycin) and/or salt or hydrate formula and/or salt or hydrate thereof; and a polymeric compound having a structure, wherein the structure is selected from the group consisting of:

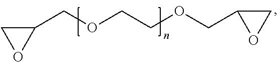

wherein n has a range of about 5 to about 135;

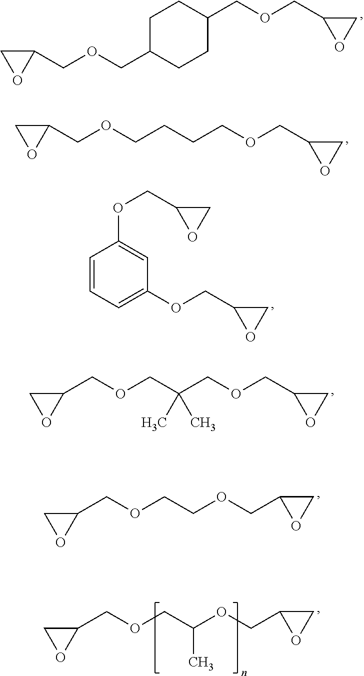

wherein n has a range of about 4 to about 15; and

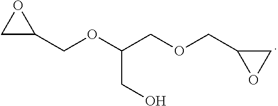 [SMILES: OCC(COCC1CO1)OCC1CO1]

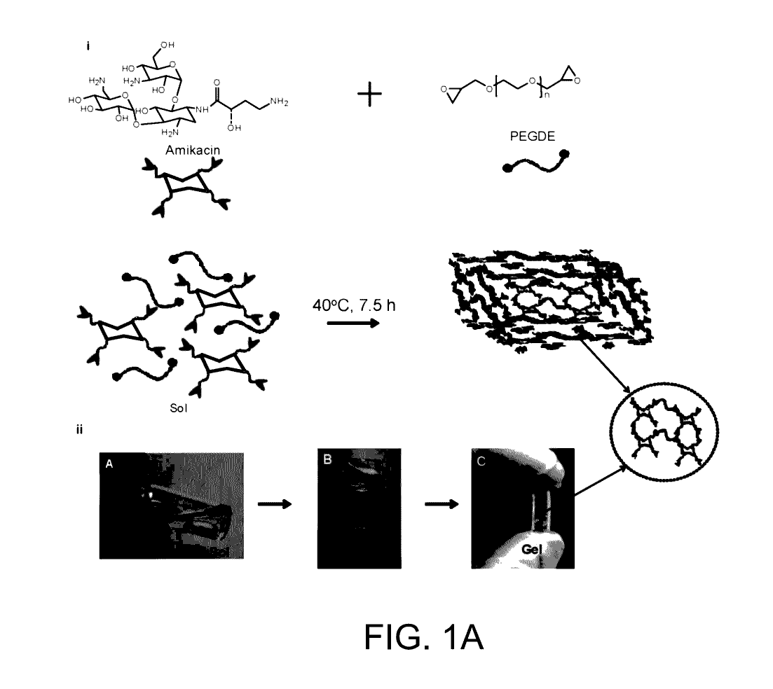

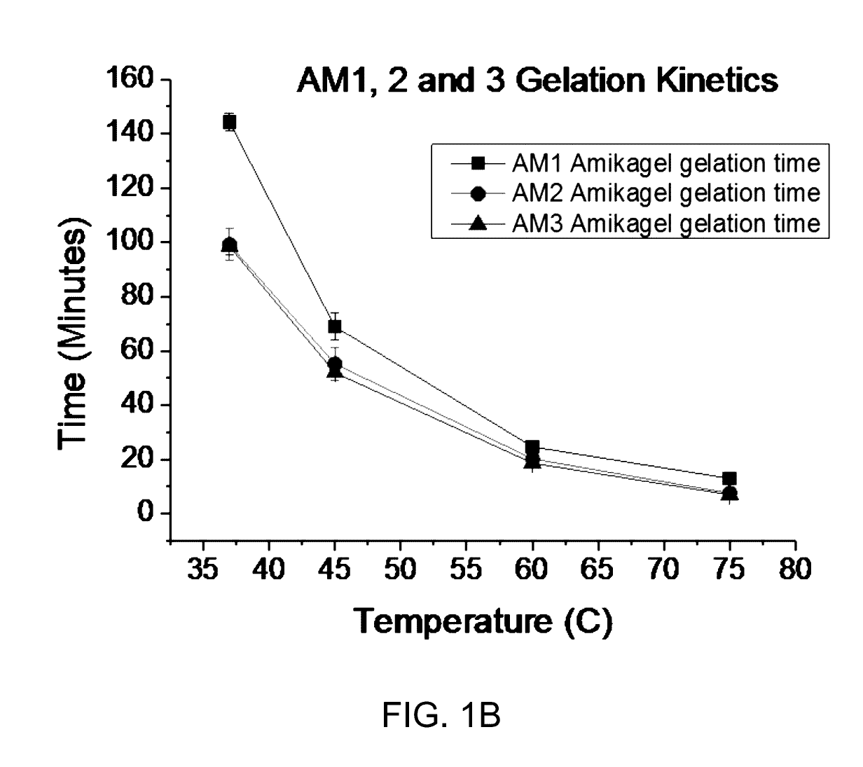

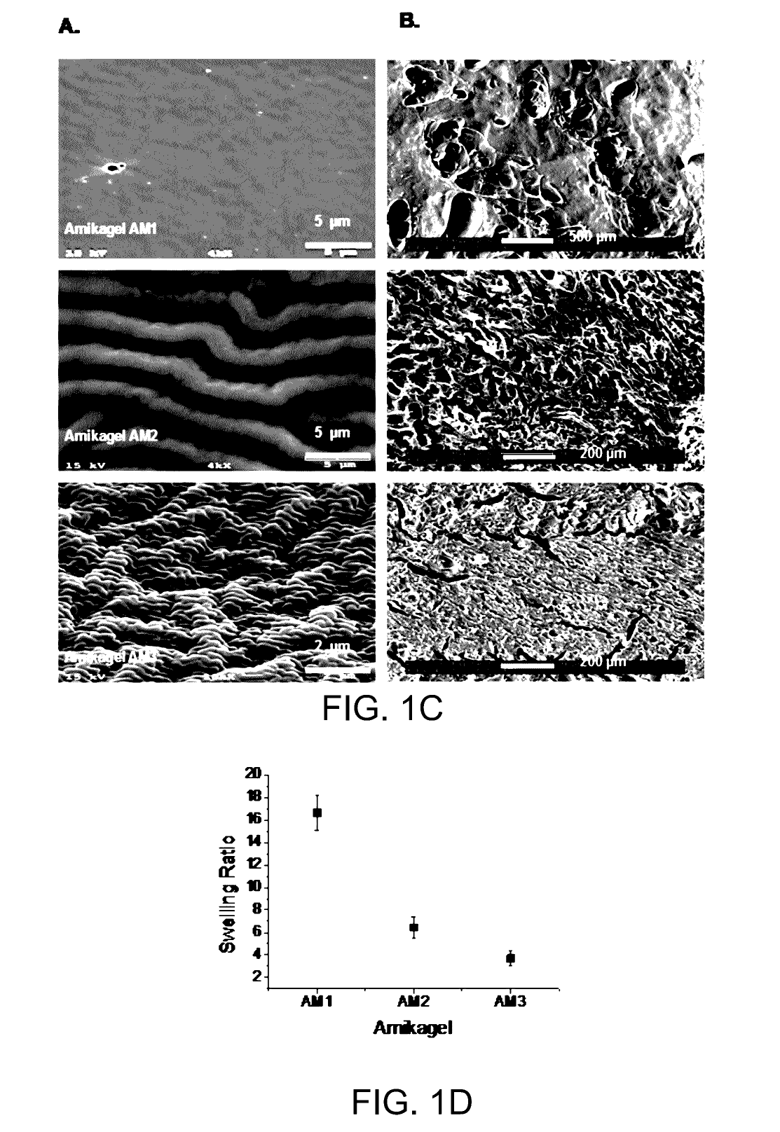

Referring to FIG. 1A, in a non-limiting exemplary embodiment, amikacin hydrate, with a molecular weight of about 603.62 and a molecular formula of C₂₂H₄₃N₅O₁₃.H₂O is used to from a cross-linked hydrogel (amikagels or AMs) with poly(ethylene glycol) diglycidyl ether (PEGDE). The number average molar mass (Mn) of PEGDE used varies. In some embodiments, the number average molar mass of PEGDE is about 500. In other embodiments, the number average molar mass of PEGDE is about 2000. In yet other embodiments, the number average molar mass of PEGDE is about 6000. As used herein, “about” is used to describe the plus or minus 10% difference in any measurement.

Different stoichiometric ratios of amikacin and the cross-linker PEGDE were dissolved in Nanopure® water, mixed and incubated at about 40° C. or a temperature that is sufficient to form the hydrogel for about 7.5 hour or for a length of time that is sufficient to form the hydrogel, in order to obtain AM1, AM2, and AM3 of different compositions. Further, the final concentration of amikacin is about 10% by weight in AM1, AM2, and AM3. In other embodiments, different final concentrations of amikacin, such as 8%-20% by weight in amikagels can be found.

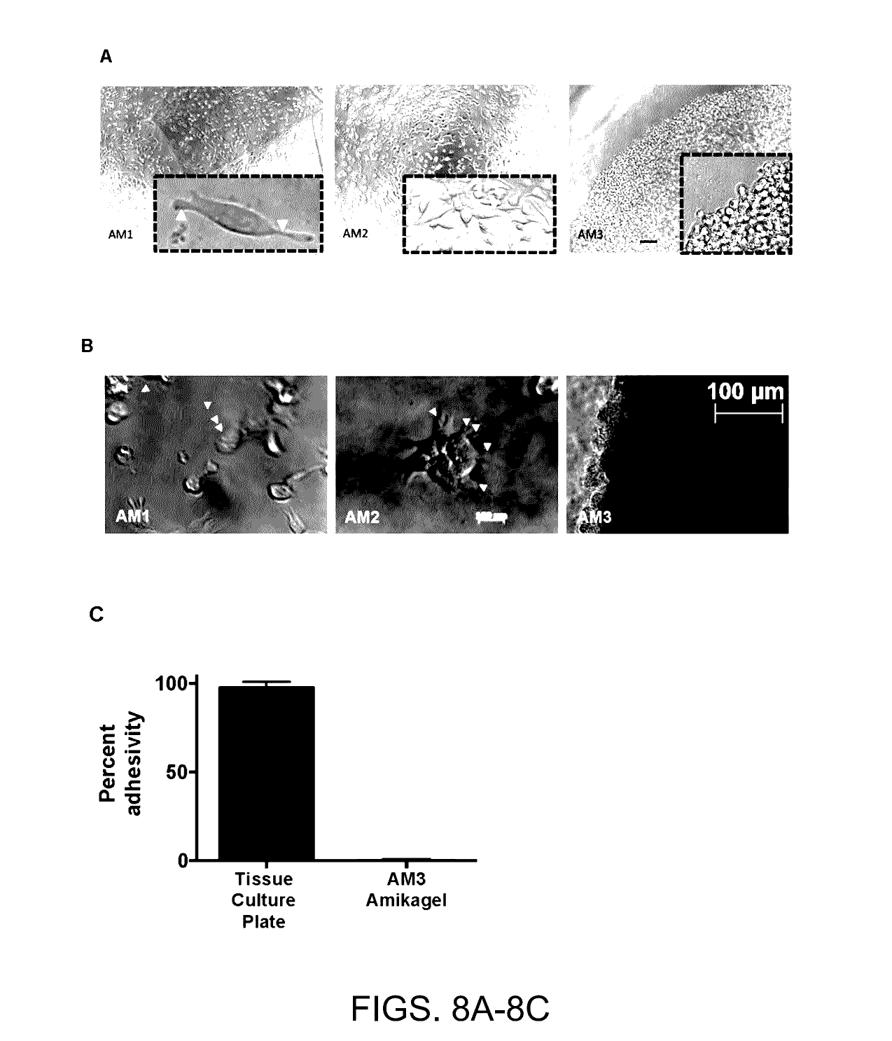

Table 1 below specifies different chemical and/or mechanical characteristics of AM1, AM2, and AM3. For example, presence of PEGDE as a cross-linker imparted amikagels with hydrophilicity, biocompatibility and a non-adherent surface chemistry. Surface adhesivity of amikagels was determined by monitoring the adhesion of bladder cancer and prostate stromal cells on the gel surface. Cells demonstrated no adhesion to the surface of AM3 amikagels, while cells attached and spread on AM1 and AM2 Amikagels. Lower amounts of amines and higher amounts of PEGDE, simultaneously engender non-adhesivity in AM3 Amikagels. For example, T24 bladder cancer cells and WPMY-1 prostate stromal cells when placed on the AM3 gel showed minimal attachment to the substrate leading to rapid spheroid formation (FIGS. 8A and 8B). In addition, qualitative measurements of adhesivity showed that AM3 gels were approximately 99.5% less adhesive compared to tissue culture plastic plate (FIG. 8C).

TABLE 1
Amikagel compositions and mechanical properties.
Hydrogel Composition

| | | Absolute Shear |
|---|---|---|
| | Mole Ratio | Modulus (KPa) |
| Amikagel | (AH:PEGDE) | (Hydrated) |
| AM1 | 1:1.5 | 7 |
| AM2 | 1:2 | 74 |
| AM3 | 1:3 | 100 |

Note:
Material stiffness or absolute shear modulus i.e. |G*| of hydrated AM1, AM2 and AM3 Amikagels are shown. An angular frequency of (0.1-62 rad/sec) and a strain of 0.1% were applied at 25° C.; storage (G′) and loss modulus (G″) of gels were recorded. Absolute shear modulus (|G*|) was calculated as (G′² + G″²)^{0.5}. AH: Amikacin hydrate; PEGDE: Poly(ethylene glycol) diglycidyl ether.

Referring to FIG. 1B, in certain embodiments, the time required for gelation at a given temperature, decreases as the amount of PEGDE increases (i.e. AM 3˜AM 2<AM 1). Further, as the gelation temperature increases, the time required for gelation reduces indicating a temperature controllable Amikagel formation process. At the highest temperature tested of 75° C., complete gelation was achieved within about 20 minutes for all the three gels. As the temperature increases, the rate of reaction between the epoxide groups of PEGDE and amine groups of amikacin moieties are likely to react rapidly leading to faster gelation. Similarly, higher amount of the cross linker will allow for faster crosslinking between the PEGDE and the amikacin groups leading to faster gelation.

Referring to FIG. 1C, macroscopic surface morphology and microscopic inner cross-linked networks of amikagels are studied using Field Emission Scanning Electron Microscopy (FE-SEM). In certain embodiments, the PEGDE content and degree of cross-linking have a significant impact on both surface and interior morphology of amikagels. The folding on the hydrogel surface, as shown in panel A, increased with increase in the degree of cross-linking. AM1 amikagel has a predominantly smooth surface, which increasingly turned into grooves and ridges as the PEGDE content increased. Internally, as the PEGDE content and degree of cross-linking increased, the pore sizes of the fractured amikagels decrease (AM3>AM2>AM1) as shown in panel B. Increase in degree of cross-linking between adjacent aminoglycoside molecules could have directly led to the reduced pore sizes and a stiffer hydrogel.

Referring to FIG. 1D, Amikagels 1, 2 and 3 exhibit swelling ratios of 16.7, 6.4, and 3.7, respectively which is similar to other PEG crosslinked hydrogel. Swelling ratios depended on the degree of cross-linking in the hydrogel network; as the degree of cross-linking increased, the swelling ratios decreased. It is likely that as the degree of cross-linking increases, gel strength increases due to the extensive crosslinks and rigidity of the network, which, in turn, prevents the gel from swelling. Increased porosity of AM1 hydrogel could have aided in increased water absorption and retention after hydration, thus increasing its swelling ratio.

Moreover, angle measurements on dehydrated Amikagels are performed in order to investigate the hydrophilicity and wettability of the surface. Contact angles (θ) of Amikagels AM1, AM2 and AM3 are found to be 52.2±1.10, 54.4±2.9° and 48.01±4.0° respectively, indicating an overall hydrophilic gel surface. Contact angle (θ) of less than 90° is considered a hydrophilic surface whereas beyond 90° constitutes a hydrophobic one. Hydrophilicity of the surface does not change with respect to the amount of PEGDE cross linker used. As amines, hydroxyls and the glycol units together contribute to the hydrophilicity, lowering the amount of one of them could be compensated by another.

After successful generation of amikagels, a three-dimensional (3D) tumor microenvironment (3DTM) is formed using the amikagels. In certain embodiments, 3DTM is generated using single cell lines of cancer cells. Different cancer cell lines can include, not limit to, T24 bladder cancer, PC3 and PC3-eGFP prostate cancer and MDA-MB-231 breast cancer cells. In other embodiments, 3DTMs are generated using co-cultures of cancer cells and stromal cells. Stromal cells can include, but not limit to, Stromal cells including NIH3T3 murine fibroblasts, BJ-5ta human foreskin fibroblasts, WPMY-1 human prostate stromal cells.

In a non-limiting exemplary embodiment, 1 ml of amikagel AM1, AM2 and AM3 pre-gel solutions were filtered through a 0.20 μm filter and 40 μl of the filtrate was added to each well of a 96 well plate. The plates were sealed with paraffin tape (Parafilm, Menasha, Wis.) and incubated in an oven maintained at about 40° C. for about 7.5 hours. After gelation, the surfaces of amikagels were washed with 150 μl of Nanopure® water for 12 hours, in order to remove traces of unreacted monomers. 3DTM experiments were set up by liquid overlay culture of cells on top of amikagel surface in a total volume of about 150 μl media/well. In certain embodiments, about 100,000 cancer cells alone (single culture) were incubated. In other embodiments, about 50,000 stromal cells followed by about 50,000 cancer cells (co-culture) were incubated. In general, a seeding density of about 1,000 to 50,000 of cancers cells are used and a seeding density of about 1,000 to 50,000 of stromal cells are used respectively. After 48 hours of incubation, 50% of the media in the wells was replaced with fresh media, i.e., DMEM/RPMI+10% (v/v) FBS+1% (v/v) Pen-Strep at regular intervals of 48 hours. Fresh media was added every 48 hours following cell plating. For 3DTM generation on 24 well plates, 400 μl of pre-gel volume was used instead of 40 μl. Different co-culture 3DTM systems are represented as fibroblast/stromal cells-epithelial cells (e.g. NIH3T3-T24, WPMY-1-T24) to accurately indicate the sequence of their addition. In certain embodiments, 3DTMs were formed 5-7 days following culture on amikagels. In other embodiments, 3DTMs containing WPMY-1 cells formed within 24 hours.

Because mechanical strength, non-adhesivity, and surface functionalization have significant impact on the formation and fate of in vitro tumor models, these characteristics of formed 3DTM are evaluated. For example, NIH3T3-T24 co-culture systems formed single 3DTMs when cultured on AM3 amikagel possesses the lowest adhesivity and maximum stiffness. Further, kinetics of 3DTM formation on amikagels was studied by imaging cells at regular intervals following seeding. The cell sheet subsequently folded upon itself resulting in the formation of the single 3DTM.

In certain embodiments, 3DTMs are formed into a spheroid structure. Spheroid formation on non-adhesive surfaces is a multi-step process. The initial phase involves an integrin based interaction between loose cells and long chain ECM molecules. After integrin mediated initial loose aggregation, E-cadherin mediated cell-cell homophilic adhesions are responsible for adjacent cell cohesion and promoting compact spheroid formation. It was noticed that NIH3T3-T24 cell co-culture formed a more compact spheroid than a spheroid with T24 cells alone. It is likely that higher quantities of ECM in presence of NIH3T3 cells lead to more compact spheroids and necrotic core formation compared to spheroids with T24 cells (alone), which did not show prominent signs of necrosis.

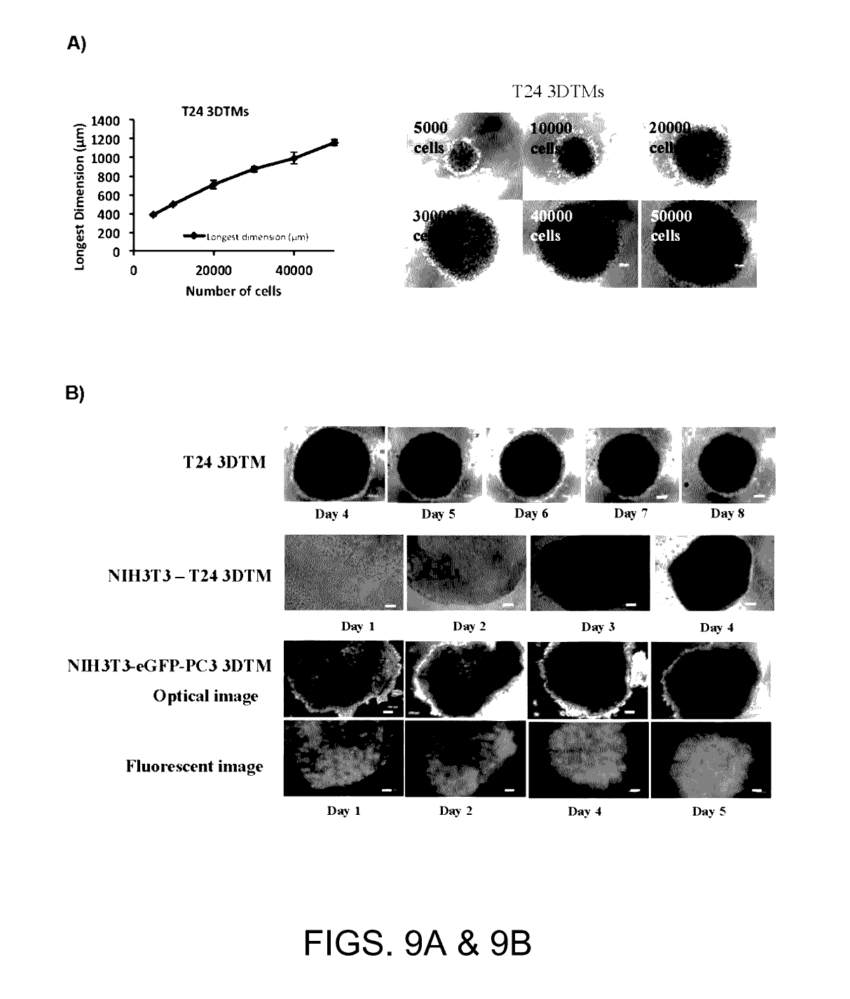

The lower mechanical strength and higher cellular adhesivity of AM1 amikagels resulted in cellular adhesion and the formation of multiple smaller 3DTMs in contrast to 3DTM formation on AM3 amikagels. T24 cells aggregated into micro-colonies (˜100 μm diameter) on day 4-5, closely resembling micro-metastatic nodules in cancer. Referring to FIGS. 9A and 9B, processes of 3DTM formation are illustrated. With more starting cells (higher cell seeding density), a larger spheroidal 3DTM is formed by measuring the longest dimension of the spheroidal 3DTM.

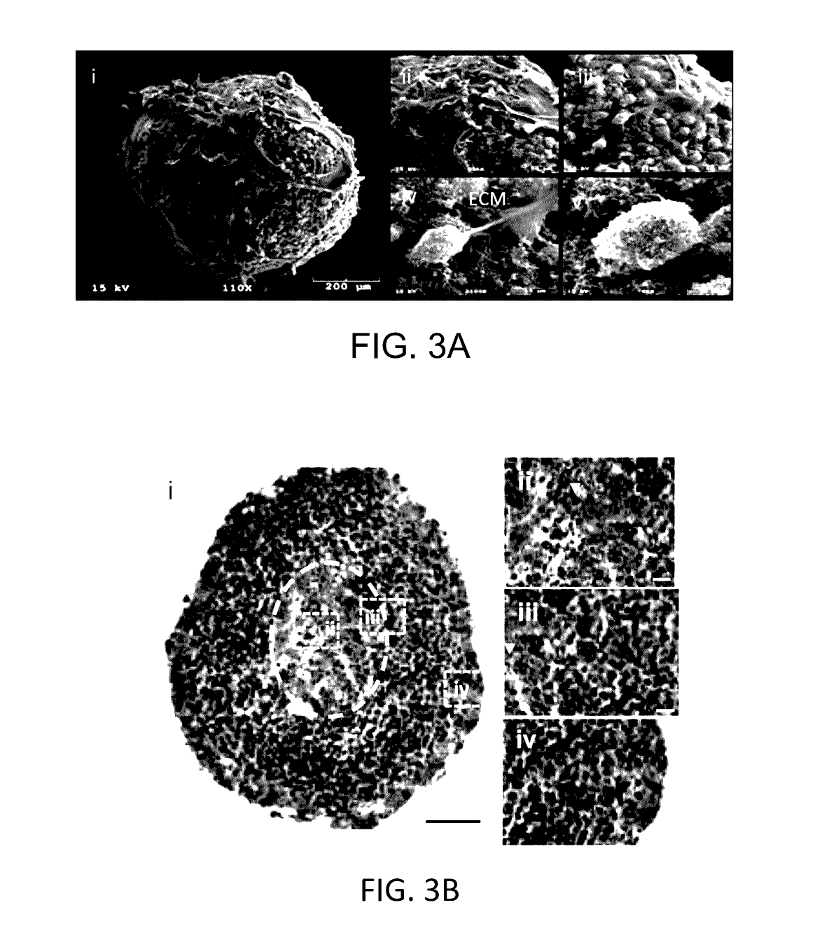

Referring to FIG. 3A, the scanning electron microscopy (SEM) imaging of NIH3T3-T24 co-cultures showed cellular attachment to each other in a compact cellular mass, covered with fibrous structures. It is likely that these fibrous structures are formed due to deposition of extracellular matrix (ECM) from the fibroblast cells present in these 3DTMs, since NIH3T3 cells are known to secrete ECM. Unlike NIH3T3-T24, those of T24 cells (alone) did not show visible presence of fibrous structures. The ability to generate ECM in 3D cell systems is significant since cancer cell-stromal cell interactions play a critical role in the local tumor microenvironment.

Now referring to FIG. 3B, 3DTMs were next analysed for histology and stress fiber formation using Hematoxylin and Eosin (H&E) and actin staining, respectively. H&E staining of NIH3T3-T24 3DTMs indicated the presence of three distinct regions: loosely packed cells in the periphery, densely packed cells in the middle, and a necrotic region in the middle that showed prominent nuclear blebbing, extensive pyknosis and absence of distinct cellular boundaries. Presence of necrotic core as a result of hypoxia and nutrient deprivation is a characteristic of tumors, and has been reported in spheroids with diameters more than 400-500 μm in diameter.

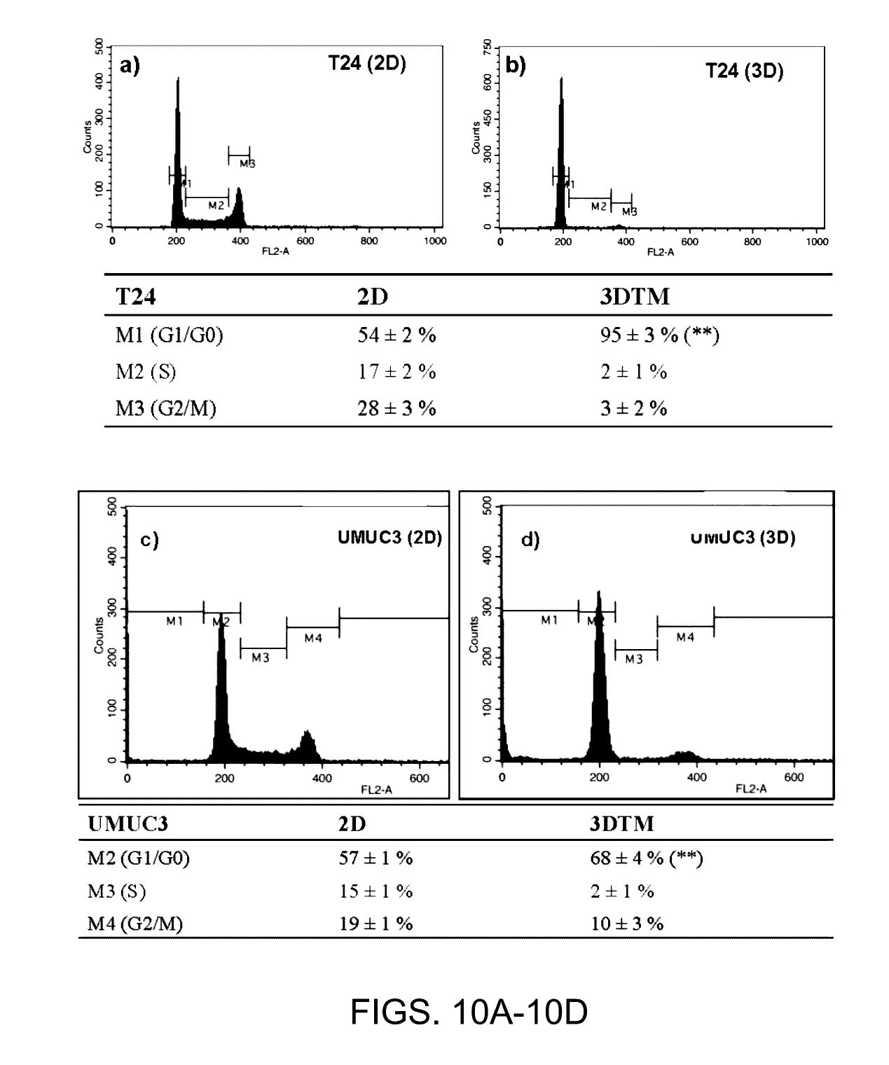

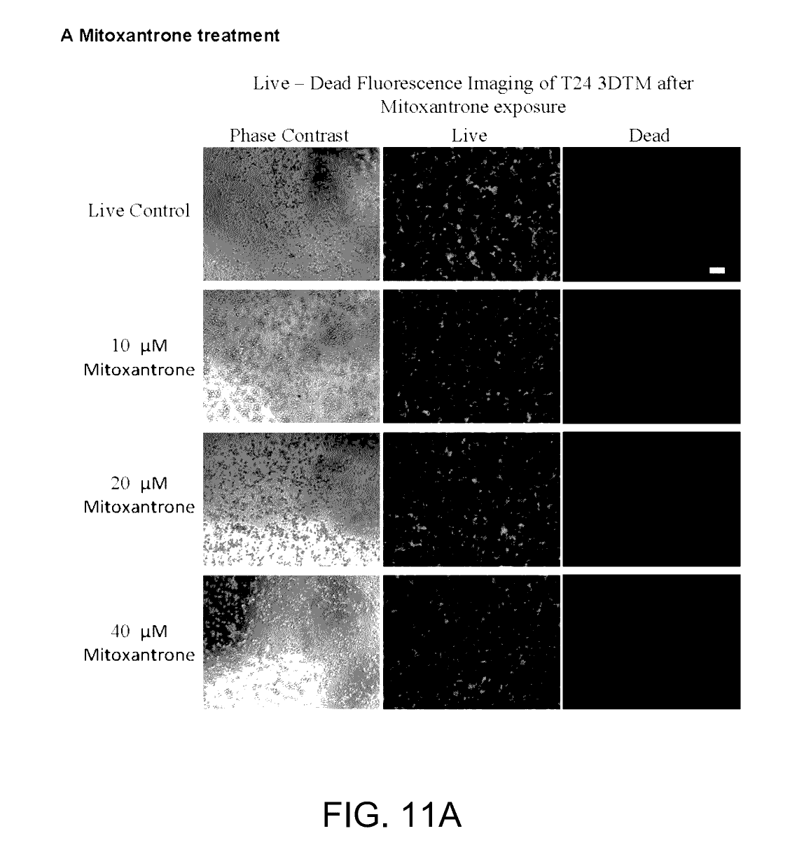

Besides sharing structural similarities of tumors, 3DTMs are also able to capture three important tumor phenotypes. For example, cell dormancy of 3DTMs are evaluated. High mechanical stress and non adhesivity (reduction in integrin signalling) have been shown to promote cell cycle arrest and in some cases, cell death via apoptosis. Referring to FIGS. 10A-10D, in certain embodiments, cell cycle analysis indicated the formation of fully dormant T24 bladder cancer 3DTMs on AM3 amikagel (about 100 kPa stiffness) and about 95% of the cell population was arrested in the G0/G1 phase of the cell cycle. In other embodiments, about 80-85% of the cell population was arrested in the G0/G1 phase of the cell cycle. In still other embodiments, about 75-80% of the cell population was arrested in the G0/G1 phase of the cell cycle. In yet other embodiments, about 70-75% of the cell population was arrested in the G0/G1 phase of the cell cycle. In yet still other embodiments, about 65-70% of the cell population was arrested in the G0/G1 phase of the cell cycle.

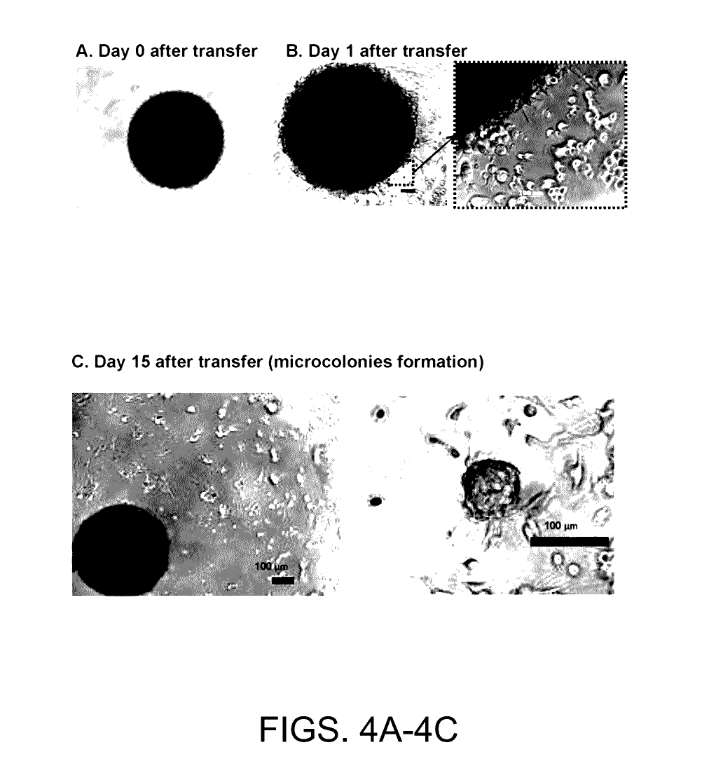

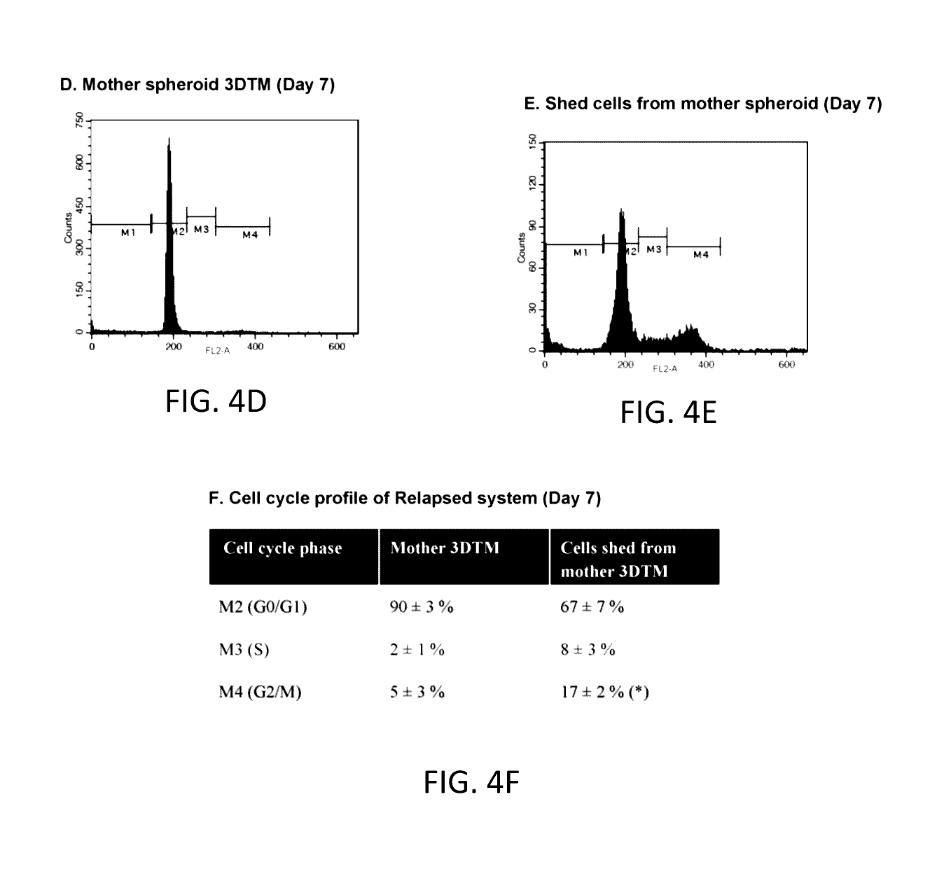

Referring to FIGS. 4A and 4B, the 3DTMs formed with amikagels are able to demonstrate escaping from tumor dormancy and forming microcolonies. In certain embodiments, T24 3DTMs generated on mechanically stiff and non-adhesive AM3 amikagels are transferred to more adhesive and mechanically weaker AM1 amikagels. Cell proliferation, spreading, and reversal of cellular dormancy upon transfer are studied. As shown in (FIGS. 4A and 4B) cells started escaping from the 3DTM within just 24 hours after transfer to AM1 amikagels. Dormant T24 3DTMs, generated on AM3 amikagels, did not demonstrate this behavior when transferred to a newly prepared AM3 amikagel, indicating the impact of the different chemomechanical microenvironment (i.e. AM1 amikagels) on tumor dormancy and relapse. At 15 days following transfer, it was clear that not all cells had left the ‘mother 3DTM’ placed on AM1 amikagels. Interestingly, cells that escaped formed microcolonies, 70-100 μm in diameter, on AM1 amikagels at significant distances away from the mother 3DTM as observed 15 days following transfer (FIG. 4C). These microcolonies can be considered to be indicative of local spread leading to distant metastases. Cell cycle studies, seven days following transfer, indicated that the ‘mother 3DTM’ continued to remain dormant (FIG. 4D), while the shed cells (FIG. 4E) demonstrated increased proliferation rates (p=0.004, two tailed t-test) (FIG. 4F). Change in media color to yellow was further indicative of proliferation in case of shed cells on AM1 amikagel.

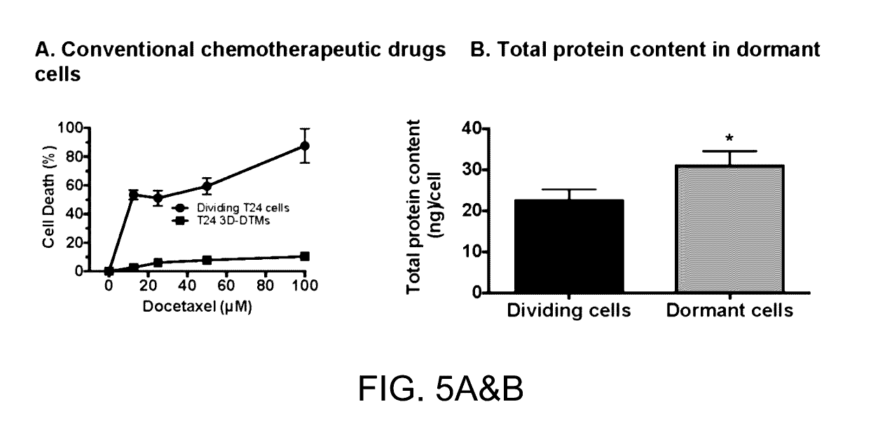

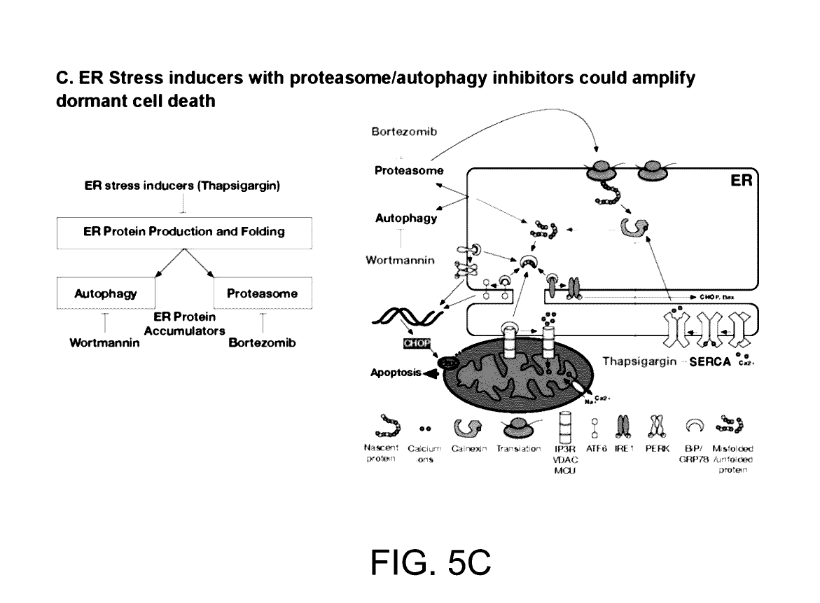

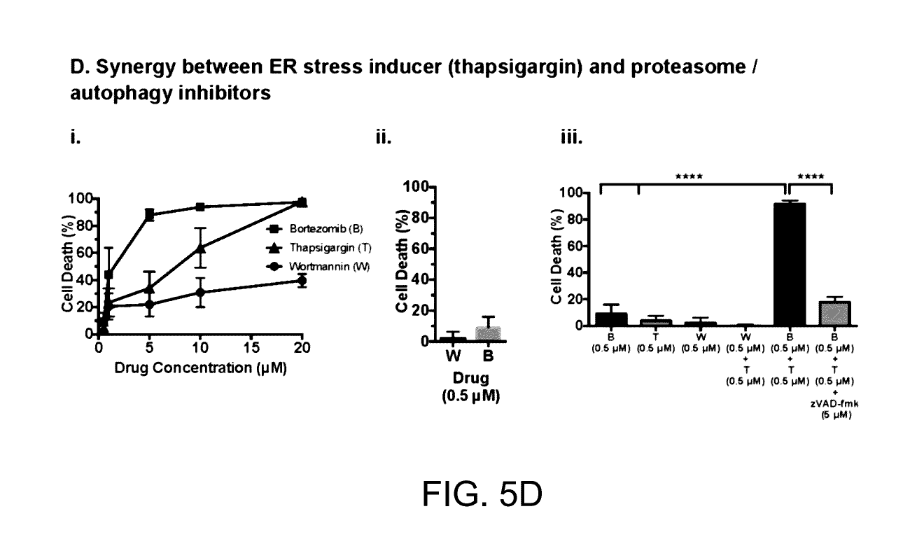

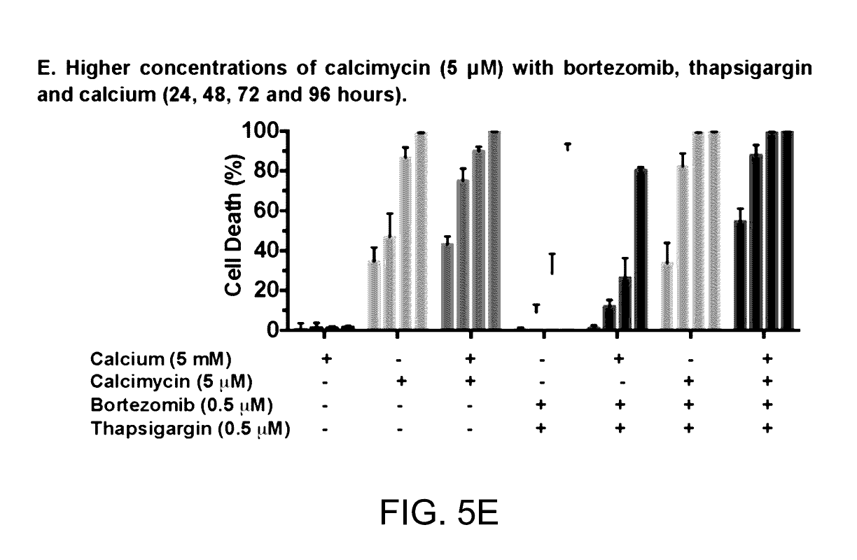

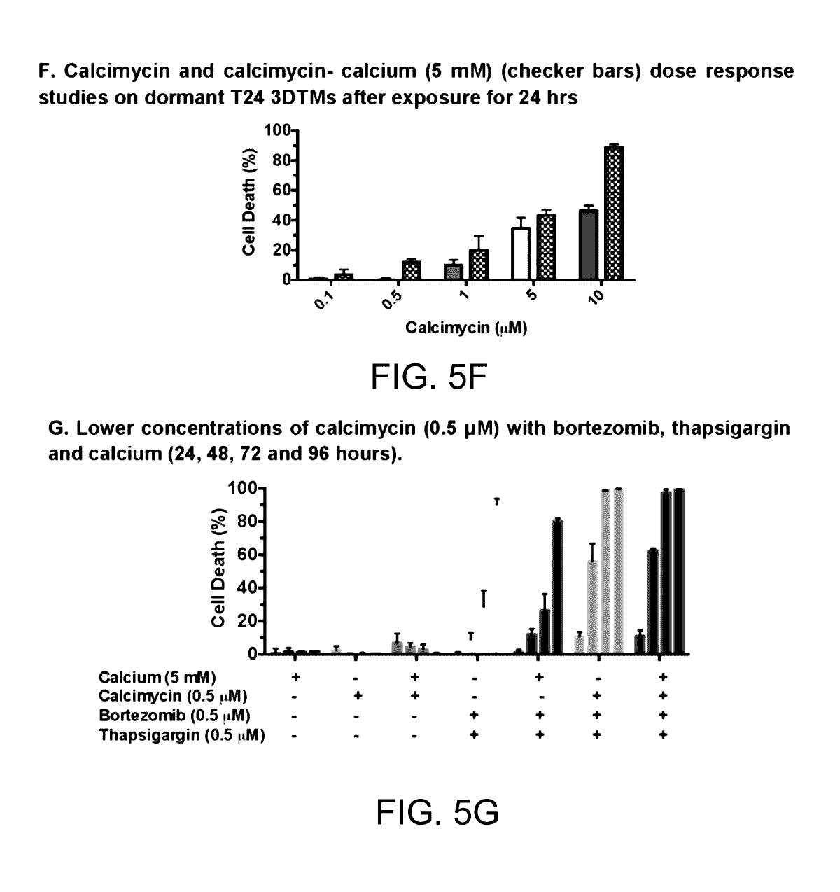

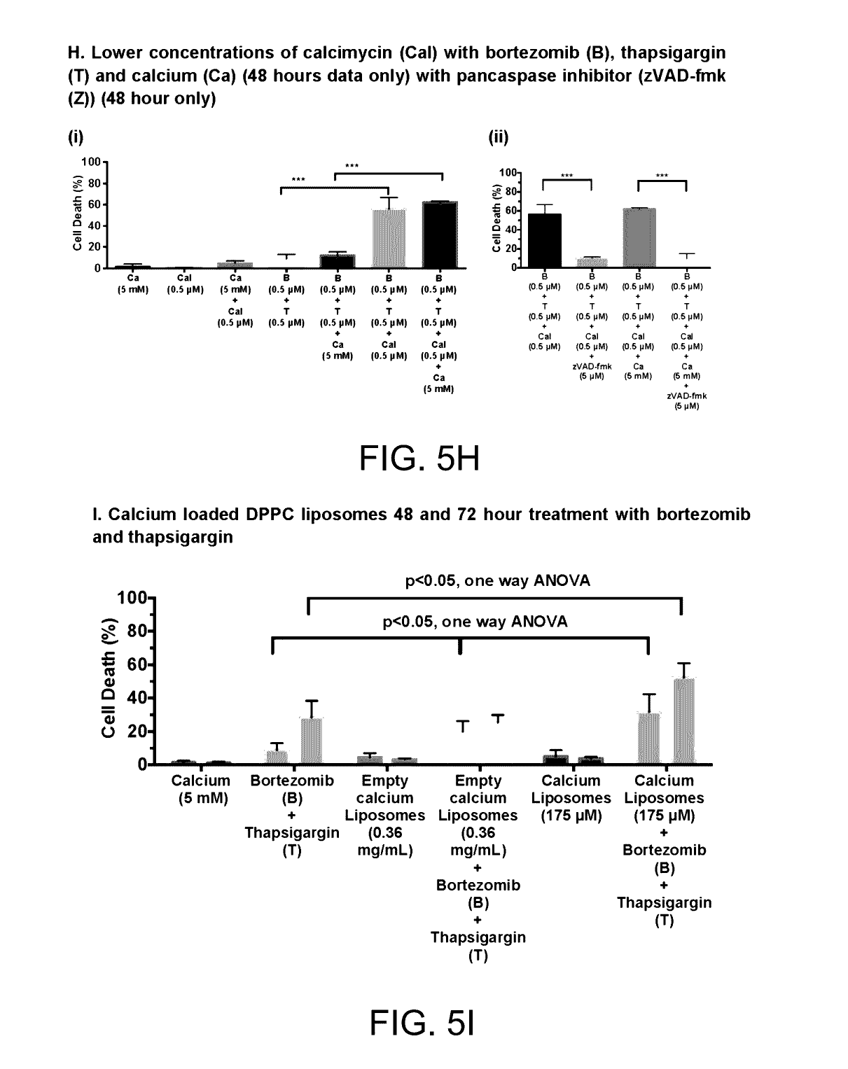

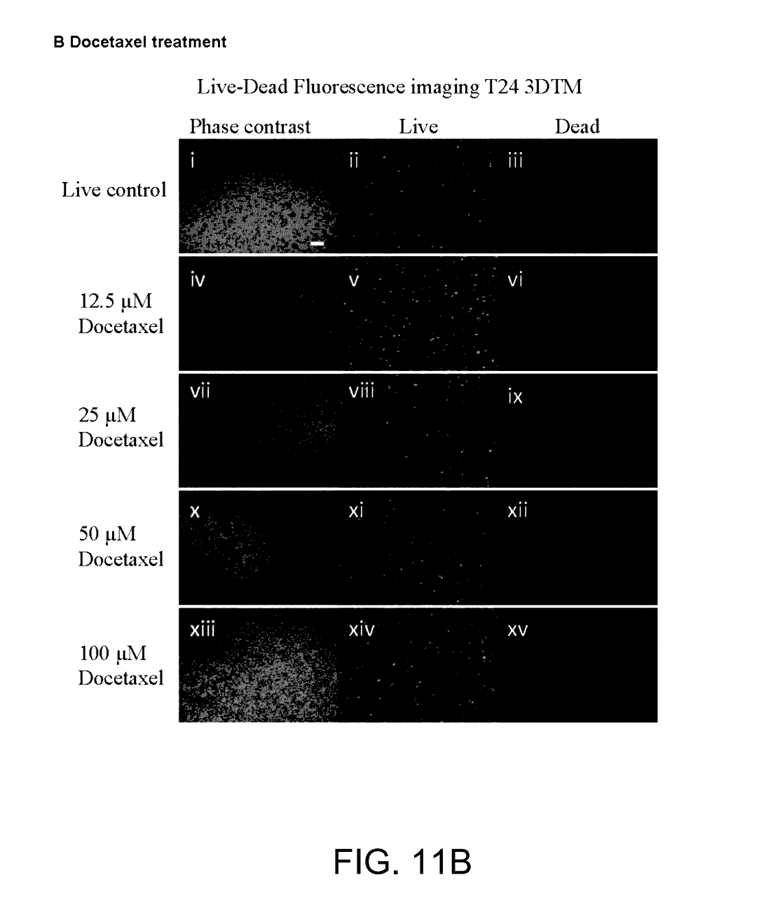

High-throughput amikagel system can be used as a drug-screening platform for identification of lead drug candidates relevant to tumor specific phenotypes. For example, this platform can be used to screen drugs for treating dormant cancer cells. 3DTMs generated with amikagels and demonstrating low sensitivity to traditional chemotherapy (FIGS. 11A-11B) serve as a platform for high throughput drug screens against the three tumor phenotypes described herein. In certain embodiments, compounds inducing endoplasmic (ER) stress is tested against said platform. FIG. 5A demonstrates that dormant tumor cells are resistant against chemotherapy and FIG. 5D illustrates a 50% induced cell death in dormant cancer cell systems with thapsigarin, which is an ER stress inducer. Further, proteasome/autophagy inhibitors show synergistic effect with ER stress inducers in inducing tumor cell death. In particular, the combination of bortezomib-thapsigargin induced nearly complete death at very low concentrations of 0.5-μM each. Now referring to FIGS. 5E and 5I, artificially raising calcium concentration in the cytoplasm accelerates cell death under chronic ER stress conditions. Further, the acceleration was reversed by pan-caspase inhibition using zVAD-fink, indicating an acceleration of apoptotic cell death during calcimycin (0.5-μM) supplementation with bortezomib-thapsigargin (FIG. 5H).

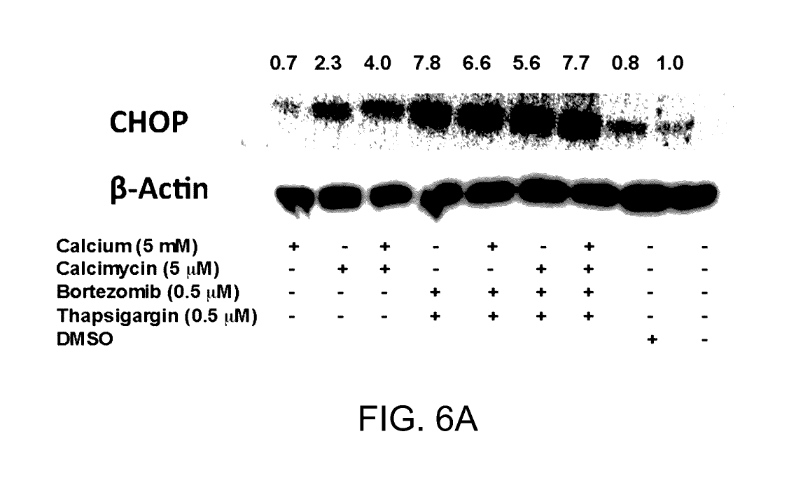

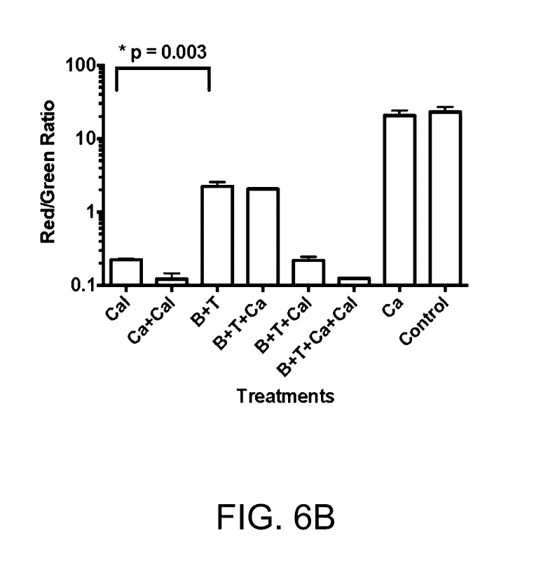

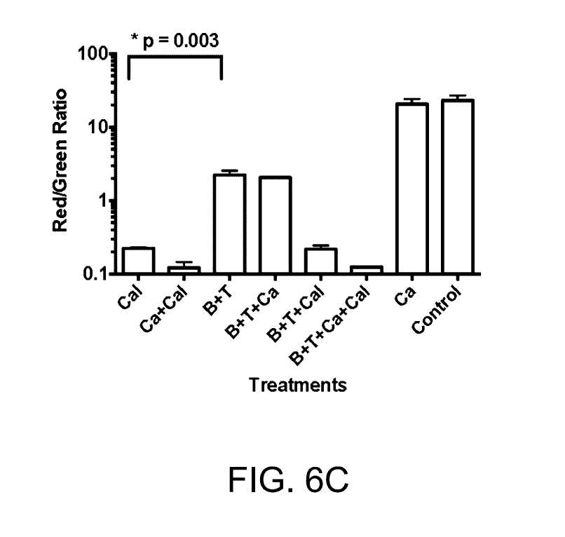

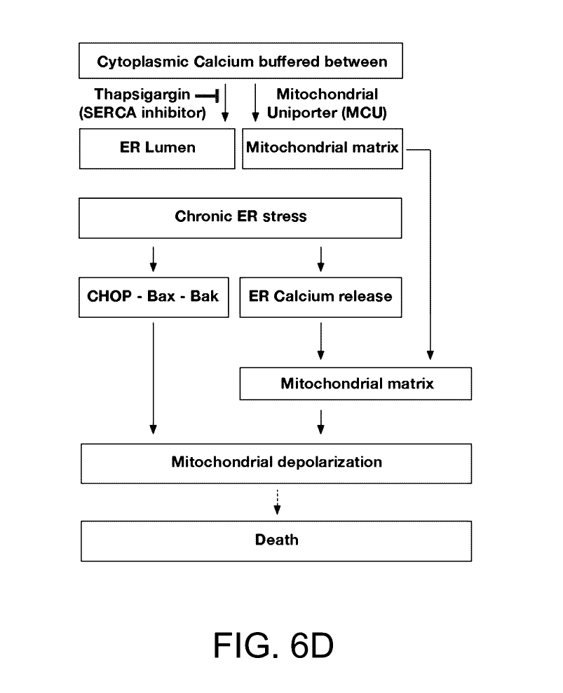

Referring to FIGS. 6A-6C, ER stress marker CHOP expression after calcium (5 mM), calcimycin (5-μM) treatment was significantly lesser than bortezomib-thapsigargin treatment and the mitochondrial depolarization and cell death % were significantly higher after 24 hours of treatment. Rapid ion influx caused by high concentration of calcimycin likely induces mitochondrial depolarization, while bortezomib-thapsigargin combination induces the depolarization via CHOP expression.

In certain embodiments, agents can be screened, including but not limited to drugs such as Cabometyx (cabozantinib), Keytruda (pembrolizumab), Lenvima (lenvatinib), Opdivo (nivolumab), Sustol (granisetron), Syndros (dronabinol oral solution), Tecentriq (atezolizumab), Venclexta (venetoclax), Alecensa (alectinib), Cotellic (cobimetinib), Darzalex (daratumumab), Empliciti (elotuzumab), Farydak (panobinostat), Ibrance (palbociclib), Imlygic (talimogene laherparepvec), Keytruda (pembrolizumab), Lenvima (lenvatinib), Lonsurf (trifluridine and tipiracil), Ninlaro (ixazomib), Odomzo (sonidegib), Onivyde (irinotecan liposome injection), Opdivo (nivolumab), Opdivo (nivolumab), Portrazza (necitumumab), Tagrisso (osimertinib), Unituxin (dinutuximab), Varubi (rolapitant), Vistogard (uridine triacetate), Yondelis (trabectedin), Akynzeo (netupitant and palonosetron), Beleodaq (belinostat), Blincyto (blinatumomab), Cyramza (ramucirumab), Imbruvica (ibrutinib), Keytruda (pembrolizumab), Lynparza (olaparib), Opdivo (nivolumab), Zydelig (idelalisib), Zykadia (ceritinib), Gazyva (obinutuzumab), Gilotrif (afatinib), Imbruvica (ibrutinib), Kadcyla (ado-trastuzumab emtansine), Mekinist (trametinib), Pomalyst (pomalidomide), Revlimid (lenalidomide), Stivarga (regorafenib), Tafinlar (dabrafenib), Valchlor (mechlorethamine) gel, Xgeva (denosumab), Xofigo (radium Ra 223 dichloride), Abraxane (paclitaxel protein-bound particles for injectable suspension), Afinitor (everolimus), Afinitor (everolimus), Bosulif (bosutinib), Cometriq (cabozantinib), Erivedge (vismodegib), Iclusig (ponatinib), Inlyta (axitinib), Kyprolis (carfilzomib), Marqibo (vinCRIStine sulfate LIPOSOME injection), Neutroval (tbo-filgrastim), Perjeta (pertuzumab), Picato (ingenol mebutate) gel, Stivarga (regorafenib), Subsys (fentanyl sublingual spray), Synribo (omacetaxine mepesuccinate), Votrient (pazopanib), Xtandi (enzalutamide), Zaltrap (ziv-aflibercept), Abstral (fentanyl sublingual tablets), Adcetris (brentuximab vedotin), Afinitor (everolimus), Erwinaze (asparaginase Erwinia chrysanthemi) Lazanda (fentanyl citrate) nasal spray, Sutent (sunitinib malate) Sylatron (peginterferon alfa-2b), Vandetanib (vandetanib), Xalkori (crizotinib), Yervoy (ipilimumab), Zelboraf (vemurafenib), Zytiga (abiraterone acetate), Halaven (eribulin mesylate), Herceptin (trastuzumab), Jevtana (cabazitaxel), Provenge (sipuleucel-T), Xgeva (denosumab), Zuplenz (ondansetron oral soluble film), Afinitor (everolimus), Arzerra (ofatumumab), Avastin (bevacizumab), Cervarix [Human Papillomavirus Bivalent (Types 16 and 18) recombinant Vaccine, Elitek (rasburicase), Folotyn (pralatrexate injection), Istodax (romidepsin), Onsolis (fentanyl buccal), Votrient (pazopanib), Degarelix (degarelix for injection), Fusilev (levoleucovorin), Mozobil (plerixafor injection), Sancuso (granisetron), Treanda (bendamustine hydrochloride), Evista (raloxifene hydrochloride), Hycamtin (topotecan hydrochloride), Ixempra (ixabepilone), Tasigna (nilotinib hydrochloride monohydrate), Torisel (temsirolimus), Tykerb (lapatinib), Gardasil (quadrivalent human papillomavirus (types 6, 11, 16, 18) recombinant vaccine), Sprycel (dasatinib), Sutent (sunitinib), Vectibix (panitumumab), Arranon (nelarabine), Nexavar (sorafenib), Alimta (pemetrexed for injection), Avastin (bevacizumab), Clolar (clofarabine), Erbitux (cetuximab), Sensipar (cinacalcet), Tarceva (erlotinib, OSI 774), Aloxi (palonosetron), Bexxar, Emend (aprepitant), Iressa (gefitinib), Plenaxis (abarelix for injectable suspension), Premarin (conjugated estrogens), UroXatral (alfuzosin HCl extended-release tablets), Velcade (bortezomib), Eligard (leuprolide acetate), Eloxatin (oxaliplatin/5-fluorouracil/leucovorin), Faslodex (fulvestrant), Gleevec (imatinib mesylate), Neulasta, SecreFlo (secretin), Zevalin (ibritumomab tiuxetan), Zometa (zoledronic acid), Campath, Femara (letrozole), Gleevec (imatinib mesylate), Kytril (granisetron) solution, Trelstar LA (triptorelin pamoate), Xeloda Zometa (zoledronic acid), Mylotarg (gemtuzumab ozogamicin), Trelstar Depot (triptorelin pamoate), Trisenox (arsenic trioxide), Viadur (leuprolide acetate implant), Aromasin Tablets, Busulflex, Doxil (doxorubicin HCl liposome injection), Ellence, Ethyol (amifostine), Temodar, UVADEX Sterile Solution, Zofran, Actiq, Anzemet, Camptosar, Gemzar (gemcitabine HCL), Herceptin, Inform HER-2/neu breast cancer test, Neupogen, Nolvadex, Photofrin, Proleukin, Sclerosol Intrapleural Aerosol, Valstar, Xeloda, Zofran, Anzemet, Bromfenac, Femara (letrozole), Gliadel Wafer (polifeprosan 20 with carmustine implant), Intron A (interferon alfa-2b, recombinant), Kytril (granisetron) tablets, Lupron Depot (leuprolide acetate for depot suspension), Miraluma test, Neumega, Quadramet (Samarium Sm 153 Lexidronam Injection), Rituxan, Taxol, Anexsia, Aredia (pamidronate disodium for injection), Arimidex (anastrozole), Campostar CEA-Scan, Elliotts B Solution (buffered intrathecal electrolyte/dextrose injection), Eulexin (flutamide), Feridex I.V., GastroMARK, Gemzar (gemcitabine HCL), Hycamtin (topotecan hydrochloride), Kadian, Leukine (sargramostim), Lupron Depot (leuprolide acetate for depot suspension), Photodynamic Therapy, Taxotere (Docetaxel), UltraJect, Visipaque (iodixanol), Zoladex (10.8 mg goserelin acetate implant), Ethyol (amifostine), Intron A (Interferon alfa-2b, recombinant), and Leukine (sargramostim).

Effects on the tumor cells are then determined, with such effects including but not limited to growth inhibition, growth arrest, induction of programmed cell death, prevention of cell migration on amenable surfaces, metastases prevention in transwell assays, reduction in cell metabolism and respiration, prevention of colony formation, prevention of tumor take in mouse models, necrosis, reduction of tumor size in orthotopic or xenografts mouse models coupled with wait gain, etc.

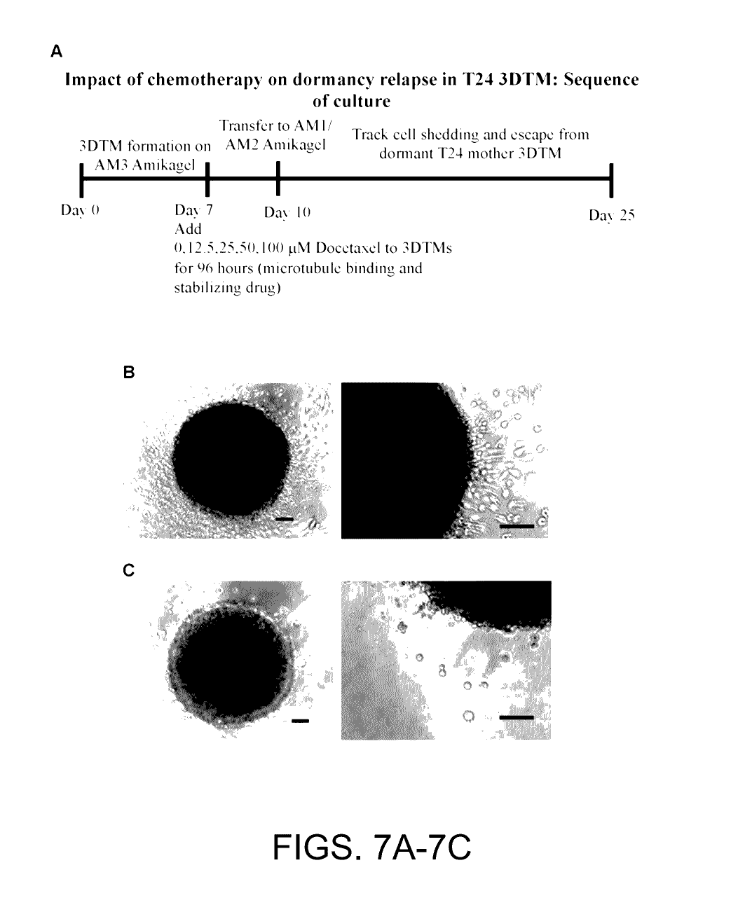

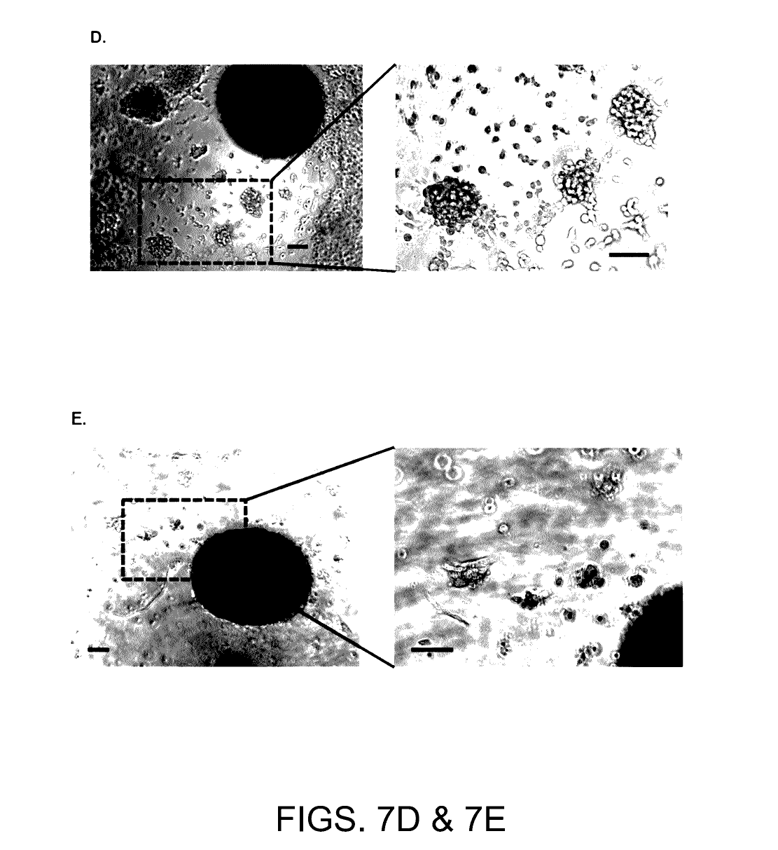

Also, high-throughput amikagel system can be used as a drug-screening platform for identification of lead drug candidates against relapse and micrometastases formation. FIGS. 7A-7E show that docetaxel treatment significantly reduced the number of cells escaping the mother 3DTM.

The following examples are presented to further illustrate to persons skilled in the art how to make and use the invention. These examples are not intended as a limitation, however, upon the scope of the invention, which is defined by claims herein. Further, every patent, patent application, and/or publication cited herein is incorporated in its entirety by reference.

### Example 1 Amikagel Formation and Characterization

Reaction of amines, present in amikacin, with epoxides in the PEGDE cross-linker resulted in the formation of a cross-linked hydrogel, amikagel (FIG. 1A) that underwent a sol-gel transition at 40° C. (FIG. 1A). FIG. 1A shows the synthesis procedure, and Table 1 shows different hydrogel compositions, AM1, AM2, and AM3, generated. amikagels AM1, AM2 and AM3 possessed material stiffness (|G*|) values of 7 kPa, 74 kPa and 100 kPa, when fully hydrated, respectively (Table 1). It was observed that |G*| values of all amikagels depended on the mole ratio of Amikacin: PEGDE added to form the hydrogel. As the amount of PEGDE relative to Amikacin increased, the absolute shear modulus (|G*|) of the gels increased (AM3>AM2>AM1), along expected lines.

Presence of poly(ethylene glycol) as a cross-linker imparted amikagels with hydrophilicity, biocompatibility and a non-adherent surface chemistry. Surface adhesivity of amikagels was determined by monitoring the adhesion of bladder cancer and prostate stromal cells on the gel surface. Cells demonstrated no adhesion to the surface of AM3 amikagels, while cells attached and spread on AM1 and AM2 amikagels. Lower amounts of amines and higher amounts of PEG (Table 1), simultaneously engender non-adhesivity in AM3 amikagels. We further investigated 3DTM formation on all three amikagels. Other aminoglycosides and crosslinkers can be used to generate aminoglycoside-based hydrogels for cell culture. The different aminoglycosides and crosslinkers that can be used to generate novel aminoglycoside based hydrogels are disclosed.

### Example 2 Generation of Three-Dimensional Tumor Microenvironments (3DTMs) on Amikagels

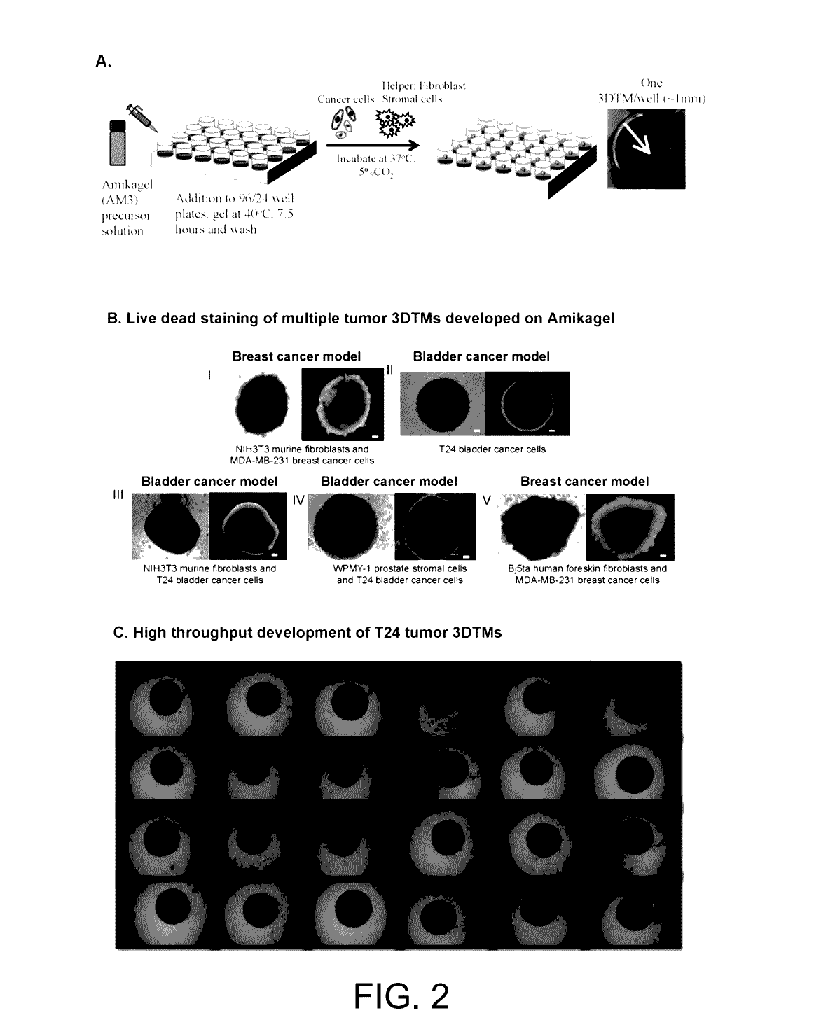

Several cancer cells, including prostate, bladder, breast, and pancreatic cancer cells, when cultured singly or with stromal/stellate/fibroblast cells on amikagel AM3 resulted in the formation of singular ˜0.8-1 mm spheroidal 3DTMs (one 3DTM per well) after 5-7 days of cell seeding FIG. 2). The size of 3DTMs could be tailored by seeding different cell densities on the AM3 Amikagel in both, 96 as well as 24 well plates; the longest dimension of the 3DTMs ranged from 300 μm to 1200 μm with increasing cell density. Co-culture of PC3-EGFP (PC3 human prostate cancer cells constitutively expressing GFP) along with red quantum dot loaded NIH3T3 fibroblasts indicated that both, cancer cells and stromal cells were present homogeneously throughout the 3DTM. The amikagel platform therefore facilitates the facile generation of different 3DTMs that can be easily adapted into a high-throughput platform. The simplicity and fidelity of this method are significant advantages over other methods, which result in the formation of heterogeneous spheroids while using more sophisticated methods including, suspension cultures using microcarrier beads in rotating wall bioreactors (11) or shaker-culture systems (12).

For the proof of concept, we demonstrated spheroidal 3DTM formation on amikagels with multiple single (T24 and UMUC3) as well as co-culture cell lines of cancer and supporting cells (NIH3T3 murine fibroblast, BJ5ta human foreskin and WPMY-1 prostate stromal cells). The rest of the studies are focused only on T24, UMUC3 and NIH3T3-T24 3DTMs.

### Example 3 Effect of Amikagel Chemo-Mechanical Properties on 3DTM Formation

Mechanical strength, non-adhesivity and surface functionalization have significant impact on the formation and fate of in vitro tumor models (13). As shown in (FIG. 2) T24 alone, and (FIG. 2) NIH3T3-T24 co-culture systems formed single 3DTMs when cultured on AM3 Amikagel, which possesses the lowest adhesivity and maximum stiffness (Table 1). Kinetics of 3DTM formation on Amikagels was studied by imaging cells at regular intervals following seeding. Cells first formed a freely floating cell sheet, approximately 1-2 cells thick, within the first two days of culture on AM3 Amikagel. The cell sheet subsequently folded upon itself resulting in the formation of the single 3DTM. Spheroid formation on non-adhesive surfaces is known to be a multi-step process. The initial phase involves an integrin based interaction between loose cells and long chain ECM molecules (14). After integrin mediated initial loose aggregation, E-cadherin mediated cell-cell homophilic adhesions are responsible for adjacent cell cohesion and promoting compact spheroid formation (14). It was noticed that NIH3T3-T24 cell co-culture formed a more compact spheroid than a spheroid with T24 cells alone. It is likely that higher quantities of ECM in presence of NIH3T3 cells lead to more compact spheroids and necrotic core formation compared to spheroids with T24 cells (alone), which did not show prominent signs of necrosis.

The lower mechanical strength and higher cellular adhesivity of AM1 Amikagels resulted in cellular adhesion and the formation of multiple smaller 3DTMs in contrast to 3DTM formation on AM3 Amikagels. T24 cells aggregated into micro-colonies (˜100 μm diameter) on day 4-5, closely resembling micro-metastatic nodules in cancer. In the study conducted by Gildea et al. (15) tumorigenic variant of T24 cells formed microcolonies on soft agar and they suggested a paracrine signaling pathway of communication between these cells activated upon mutual contact. We believe the T24 cell population is a heterogeneous mix of invasive and non-invasive cancer cells of differential HRAS expression (15); invasive cells allow the formation of these microcolonies beyond confluency. Our other studies below support this hypothesis.

### Example 4 2.4. Morphological and Biochemical Characterization of 3DTMs

Scanning electron microscopy (SEM) imaging of NIH3T3-T24 (FIG. 3A) co-cultures showed cellular attachment to each other in a compact cellular mass, covered with fibrous structures. It is likely that these fibrous structures are formed due to deposition of extracellular matrix (ECM) from the fibroblast cells (16) present in these 3DTMs (FIGS. 3A-B), since NIH3T3 cells are known to secrete ECM (17). Unlike NIH3T3-T24, those of T24 cells (alone) did not show visible presence of fibrous structures. The ability to generate ECM in 3D cell systems is significant since cancer cell-stromal cell interactions play a critical role in the local tumor microenvironment (18).

Following formation, 3DTMs were next analyzed for histology and stress fiber formation using Hematoxylin and Eosin (H&E) and actin staining, respectively. H&E staining of NIH3T3-T24 3DTMs indicated the presence of three distinct regions: loosely packed cells in the periphery, densely packed cells in the middle, and a necrotic region in the middle that showed prominent nuclear blebbing, extensive pyknosis and absence of distinct cellular boundaries (FIG. 3B). Presence of necrotic core as a result of hypoxia and nutrient deprivation is a characteristic of tumors, and has been reported in spheroids with diameters more than 400-500 μm in diameter (19). Presence of a necrotic core in NIH3T3-T24 3DTM could be associated to the presence of dense extracellular matrix (ECM) in the spheroid (FIGS. 3A-B). Presence of high amounts of ECM in the tumor often constitutes a fibrosis response that can prevent free diffusion of nutrients, metabolites and cause necrosis (21). Interestingly, unlike NIH3T3-T24 3DTMs, T24 3DTMs (7 day) by themselves did not demonstrate a distinct necrotic core region. It is likely that the absence of ECM deposition in T24 3DTMs is responsible for low compaction of cells and easier access of nutrients throughout the spheroid. This, in turn, results in the absence of a distinct necrotic core in T24 3DTMs.

Previous results with Live/Dead® staining of 3DTMs generated using 3T3 murine fibroblasts indicated an outer green (viable) ‘ring’ with an inner red (dead/dying) core, indicating a metabolically active and viable outer shell of cells, along with a stressed inner core of cells (FIG. 2). However, 3DTMs generated using T24 cells did not show prominent red-staining in the core, indicating differential biochemical consequences depending on the cells employed (FIG. 2). The H&E results are consistent with those observed with H&E staining, and indicate that the outer cells layers are alive while inner cell layers are metabolically inactive/stressed in 3DTMs (FIG. 3B).

Actin staining of 40 μm thick T24 3DTM cryosections indicated that F-actin (red stain) was localized along the intracellular cortical regions in T24 3DTMs. Unlike 2D cell culture plate wherein cellular F-actin stress fibers support strong cell-substratum interactions (22), localization of F-actin filaments in the cellular cortex in T24 3DTMs might indicate presence of cell-cell interactions, rather than cell-substratum interactions.

### Example 5 2.5 Investigation of 3DTM Dormancy on Amikagels

High mechanical stress and non adhesivity (reduction in integrin signalling (23)) have been shown to promote cell cycle arrest and in some cases, cell death via apoptosis. For example, Cheng et al. reported decrease in hepatocellular spheroid viability when grown on stiffer agarose hydrogels compared to gels with lower stifffness (13). In the case of our 3DTMs, cell cycle analyses (Table 2) indicated the formation of fully dormant T24 bladder cancer 3DTMs on AM3 Amikagel (˜100 kPa stiffness); ˜95% of the cell population was arrested in the G0/G1 phase of the cell cycle. This is in stark contrast to the cell cycle profile of 2D culture of T24 cells in which, ˜54% cells were in the G0/G1 phase of the cell cycle. UMUC3 bladder cancer cells also had a similar response (cell cycle arrest) to spheroid formation, although the percentage of cells arrested in the G0/G1 phase were not as high as T24 cells. T24 cells are known to be contact inhibited, due to the upregulation of p27 CDK inhibitor upon confluency (24). Aggregation of cells with each other into the 3DTM could have resulted in a response similar to contact inhibition. Barkan et al. (23) showed that dormant breast cancer D2.0R tumors have high expression of p27 (˜77% of cell nuclei) and lower amounts of (˜50%) of p16. These dormant D2.0R breast cancer cells when injected into mice invaded distant metastatic sites and remained as single quiescent cells for prolonged periods of time before metastases. Near complete total dormancy achieved on Amikagel platform allows its usage as a clinically relevant model for high-throughput drug discovery.

TABLE 2
Cell cycle profile on Amikagel AM3

| Cell line | Cell cycle phase | 2D culture | 3DTM |
|---|---|---|---|
| T24 bladder | M1 (G1/G0) | 54 ± 2% | 95 ± 3% (**) |
| cancer cellls | M2 (S) | 17 ± 2% | 2 ± 1% |
| | M3 (G2/M) | 28 ± 3% | 3 ± 2% |
| UMUC3 bladder | M1 (G1/G0) | 57 ± 1% | 68 ± 4% (**) |
| cancer cells | M2 (S) | 15 ± 1% | 2 ± 1% |
| | M3 (G2/M) | 19 ± 1% | 10 ± 3% |
| NIH3T3-T24 | M1 (G1/G0) | — | 82 ± 2% |
| Co-culture 3DTM | M2 (S) | — | 11 ± 4% |
| | M3 (G2/M) | — | 4 ± 1% |

Cell cycle analysis: summary table showing cell population in 2D vs 3D cultures for different bladder cancer cell lines as individual and co-cultures. ** indicates p<0.0001 for G0/G1 population compared for 2D and 3DTM of T24 and UMUC3 bladder cancer, respectively. Statistical significance determined using Student's t-test of at least n=3 independent experiments.

T24 dormant cells also showed reduced metabolic consumption compared to the actively dividing T24 cells. We also compared the effectiveness of Amikagel with non-adhesive agarose gel in spheroid formation and inducing total dormancy in T24 cells. As shown in, T24 cells cultured on agarose gel of similar mechanical properties showed a significantly different cell cycle profile and spheroid morphology compared to Amikagel. 1% agarose gel (˜7 kPa stiffness) induced spheroid formation, unlike AM1 gels which predominantly caused formation of smaller microcolonies. 10% agarose gels (˜100 kPa stiffness) agarose gel induced predominant cell death in the T24 cells, unlike AM3 Amikagels that induced dormant spheroid formation. Spheroids formed on 1% agarose gel had significantly lesser number of cells arrested in the G0/G1 phase compared to spheroids formed on AM3 Amikagels. We noticed that non-confluent T24 cells on AM1 gels had a higher percentage of cells in the G2/M phase, which reduced significantly upon confluency (microcolony formation) (p<0.05, Student's t-test). It is likely that, for agarose gels even though the mechanical stiffness match to that of Amikagels, the non-adhesivity of the two substrates could be different, thus producing a differentiated response. T24 cells cultured on matrigel showed significant differences in media consumption compared to dormant cultures. In addition, the T24 cells on matrigel formed multiple clusters unlike those seen on agarose or amikagels.

Arrest of cells in the G0/G1 phase of the cell cycle is one of the characteristics of tumor dormancy (5), and leads to resistance against chemotherapeutics that are particularly effective against rapidly dividing cells. Cell cycle profile of NIH3T3-T24 co-culture 3DTMs indicated that a majority of the cell population was also arrested in G0/G1 and S phase of cell cycles. The arrest of NIH3T3-T24 3DTM cells in non-mitotic phases of cell cycle could create resistance to traditional chemotherapy.

These results are of significance since very few methods have been developed for generating high-throughput 3D models of tumor cell dormancy; most methods using 3D models demonstrate ˜70% cells in the G0/G1 phase of the cell cycle for cancer cells at best (25). Increasing evidence is pointing towards a definitive phase of quiescence (prolonged G0/G1 arrest) rather than balanced proliferation to support prolonged viability before relapse (26). Our ability to capture bladder carcinoma dormancy on a high-throughput Amikagel platform is very useful for large drug screens for drug discovery against these cancer phenotypes. Complete dormant cells in the form of spheroids of different sizes, enable their easy recovery and transplantation into multiple animal models in the actual site of bladder (for transitional cell carcinoma), which is not possible with 2D cell cultures or single cells (require subcutaneous injection). We propose Amikagels as a novel platform for drug screening that can capture important tumor dormancy in a 96 well plate format.

### Example 6 Engineering Relapse from Dormancy in 3DTMs by Modulating Amikagel Chemomechanics

### 2.6.1 Escape from Tumor Dormancy and Microcolony Formation

Escape from dormancy is a hallmark of several cancer diseases in which, tumors, either at the primary site or at distant metastatic sites, revert to a more aggressive and proliferating phenotype, often resulting in patient mortality (5). Previous research has shown that cellular quiescence can be reversible depending upon the microenvironment surrounding the cells. Barkan et al. (23) showed that dormancy of quiescent D2.0R breast cancer cells could be reversed by supplementing fibronectin as the extracellular matrix. Fibronectin supplementation induced β1 integrin mediated signaling, which, in turn, led to cell proliferation. We next asked if modulation of the chemomechanical properties of Amikagels can result in relapse in the case of the 3D tumor models. Specifically, we transferred T24 3DTMs generated on mechanically stiff and non-adhesive AM3 Amikagels to more adhesive and mechanically weaker AM1 Amikagels, and investigated cell proliferation, spreading, and reversal of cellular dormancy upon transfer. As shown in (FIGS. 4A, B) cells started escaping from the 3DTM within just 24 hours after transfer to AM1 Amikagels. Dormant T24 3DTMs, generated on AM3 Amikagels, did not demonstrate this behavior when transferred to a newly prepared AM3 Amikagel, indicating the impact of the different chemomechanical microenvironment (i.e. AM1 Amikagels) on tumor dormancy and relapse. At 15 days following transfer, it was clear that not all cells had left the ‘mother 3DTM’ placed on AM1 Amikagels. Interestingly, cells that escaped formed microcolonies, 70-100 μm in diameter, on AM1 Amikagels at significant distances away from the mother 3DTM as observed 15 days following transfer (FIG. 4C). These microcolonies can be considered to be indicative of local spread leading to distant metastases. Cell cycle studies, seven days following transfer, indicated that the ‘mother 3DTM’ continued to remain dormant (FIG. 4D), while the shed cells (FIG. 4E) demonstrated increased proliferation rates (p=0.004, two tailed t-test) (FIG. 4F). Change in media color to yellow was further indicative of proliferation in case of shed cells on AM1 Amikagel.

It is likely that only the invasive T24 bladder cells from the population leave the mother spheroid and invade AM1 Amikagel. In studies by Makridakis et al. (27), metastatic T24 cell line was obtained by subcutaneously injecting 106 cells into the flanks of 15 adult male SCID mice. Of these only 5 mice bore tumorigenic outgrowths of T24 cells, while others did not, which were harvested and propagated for experiments. It is plausible that only the cells received by those 5 mice contained highly tumorigenic cells in them at higher densities. These aggressive T24 cell lines had increased proteasome activity, lower CATD (cathepsin D) activity (a poor prognostic factor of bladder cancer (28)). Research by Gildea et al. (15) showed higher expression of HRAS in metastatic version of T24 cells which leads to focal adhesion disassembly, loss of β-catenin and invasion. In addition, T24 cells are known to be E-cadherin null and it is most likely that N-cadherin is associated with β-catenin (29), which makes our heterogeneous cell escape and their microcolony formation results very interesting. Detailed analyses of the differences between the mother spheroid and the escaped cells is beyond the scope of this paper and will be followed up in subsequent studies.

Dormant T24 3DTMs placed on AM3 Amikagels recorded a decrease in their size, likely due to senescence, despite no obvious visual observation of cell escape. It has been shown before that extensive periods of externally induced cell cycle arrest in presence of growth stimulation (fetal bovine serum addition) can lead to cellular senescence (30). Taken together, our results indicate that modulating Amikagel chemo-mechanical properties can result in both, 3DTM models of (1) tumor dormancy, (2) cellular escape from dormancy, and (3) formation of micrometastasis on a high-throughput scale.

### Example 7. Amikagel Platform as High-Throughput Screen of Chemotherapy Against Tumor Specific Phenotypes

After three important tumor phenotypes were captured on Amikagels, we used Amikagel-3DTM platform for high throughput drug screens against those specific phenotypes. T24 (alone), and NIH3T3-T24 3DTMs were exposed to different doses of the DNA-damaging drug, mitoxantrone (31) or the microtubule-stabilizing drug, docetaxel for 96 hours (32). Co-culture 3DTMs were treated with collagenase to allow their disassembly before cell-viability measurement. It was noticed that collagenase treatment enabled very easy diassembly of the co-culture 3DTMs, supporting our previous finding of ECM production.

It was observed that NIH3T3-T24 3DTMs were resistant to mitoxantrone for doses as high as 80 μM. We verified that lack of cell death was not due to poor drug penetration into the 3DTM; fluorescence microscopy indicated mitoxantrone permeation throughout the NIH3T3-T24 3DTM in 24 hours. The LC50 concentration (dose at which 50% of the cells lose their viability) of mitoxantrone was approximately 9 μM in 2D cultures of T24 cells (our previous data (31)). NIH3T3-T24 3DTMs were also resistant to the alkylating agent ThioTEPA up to a concentration as high as 500 μM. The arrest of NIH3T3-T24 3DTM cells in non-mitotic phases of cell cycle correlates to its chemotherapeutic resistance towards different anticancer drugs such as thioTEPA and mitoxantrone despite complete drug penetration.

Similar results of chemotherapeutic resistance were obtained when T24 3DTMs were treated with mitoxantrone and docetaxel. Treatment with doses as high as 100 μM docetaxel resulted in only ˜10% death (compared to live control) even after 96 hours of exposure (FIG. 5A). In contrast, the docetaxel LC50 concentration (drug dose that causes 50% cell death) for T24 cells was determined to be 10 μM in 2D culture (FIG. 5A), indicating the significant resistance of these 3DTMs to chemotherapeutic treatment. Cellular dormancy of T24 3DTMs was reflected in the lack of their susceptibility to docetaxel and mitoxantrone (FIG. 5A). In NIH3T3-T24 cells, ECM-mediated resistance, in concert with cellular arrest, could be responsible for the dormancy and chemotherapeutic resistance of 3DTMs (33). It is important to note that resistance to chemotherapeutics is not due to poor transport of drugs into 3DTMs; microscopy studies indicated that despite complete penetration of mitoxantrone, 3DTMs were resistant to the drug.

Low sensitivity to traditional chemotherapy is a hallmark of cancer resistance and/or dormancy, particularly in advanced cases. Tumor dormancy is a significant clinical challenge, with very few in vitro tools that facilitate investigations into the underlying biology and high-throughput drug discovery. Our results indicate that 3DTMs generated on Amikagels demonstrate significant resistance to traditional chemotherapeutics, and are ideal for studying tumor dormancy and resistance.

### Drugs Against Dormancy

After showing remarkable resistance to traditional chemotherapies of mitoxantrone and docetaxel, we explored for other alternatives to induce cell death in dormant spheroids generated using Amikagel platform. We used Amikagel platform to identify new drug regimens against tumor specific phenotypes. Inducing death in dormant stage of cancer is necessary to avoid their relapse in future. During the characterization of dormant tumor phenotype, we observed a slight but significantly higher per cell protein content in dormant T24 cells when compared to actively dividing T24 cells on day 7 of culture (FIG. 5B). Cellular arrest in G0/G1 phase of cell cycle has been associated with increase in total protein content in cells (34).

In order to exploit the higher per cell protein content, we hypothesized that ER stress inducers that cause protein misfolding could sensitize the dormant cancer cells to death via the unfolded protein response (UPR) and chronic ER stress (FIG. 5C). We used thapsigargin (SERCA, ER calcium channel inhibitor) to induce ER stress. Thapsigargin, an ER specific calcium channel blocker, is known to reduce calcium concentration in the ER-lumen, causing malfunctioning of calcium dependent ER chaperones (35). Blockade of ER calcium entry also elevates the cytoplasmic calcium concentration. As shown in FIG. 5D, ER stress inducer thapsigargin (ER calcium depletory drug that causes protein misfolding via calcium dependent chaperone inhibition) induced 50% death at 15-μM concentration, a significantly better response than conventional chemotherapeutic drugs such as docetaxel and mitoxantrone. In order to reduce the drug load of ER stress inducers, we explored their synergy with proteasome and autophagy inhibitors (bortezomib and wortmannin).

Under ER stress, cells can utilize protein degradation pathways via proteasome or autophagy to remove misfolded/unfolded proteins (36, 37). We therefore hypothesized that ER stress inducers, in concert with proteasome/autophagy inhibitors could impede ER-associated misfolded protein degradation, cause misfolded protein accumulation, chronic ER stress and synergistically induce death of dormant cancer cells (FIG. 5C). Chronic ER stress is known to induce cell death via mitochondrial depolarization (38), which acts as a significant amplifier of cell death during the stress (39). In addition, mitochondrial depolarization acts as a point of no return for cellular apoptosis (40). In a well-elucidated pathway (FIG. 5C) (38, 39), accumulation of misfolded proteins in ER causes the activation of two main pathways, the Irel/XBP-1, ATF6-dependent pathway and PERK/eIF-2alpha phosphorylation-dependent pathway which activate chaperone and pro-apoptotic protein production. Cell death is achieved by upregulation of pro-apoptotic transcription factor such as CHOP and Bax that punctures mitochondrial outer membrane. In addition, intra-ER calcium is released into mitochondrial matrix via specialized IP3R-VDAC-MCU channels during prolonged ER stress (FIG. 5C) (39). The calcium pumped into the mitochondrial matrix depolarizes it, leading to swelling, rupture and eventually apoptosis (39). We believe that misfolding of the higher protein content in dormant cells could provide a stronger UPR response compared to actively dividing ones. Our results using single agent proteasome/autophagy inhibitory drugs showed that single agent autophagy inhibition using wortmannin induces significantly (p<0.05, two tailed Student's t-test, concentrations 5-20 μM) lower cell death compared to single agent bortezomib (FIG. 5D).

To compare the synergistic effect of thapsigargin (ER stress inducer) with proteasome/autophagy inhibitors (bortezomib and wortmannin), 0.5-μM concentrations of the drugs (bortezomib and wortmannin) were chosen. As shown in FIG. 5D, the single agent toxicity of 0.5-μM wortmannin and bortezomib were similar to each other (<10% cell death). As shown in FIG. 5D, addition of bortezomib (proteasome inhibitors) synergistically improved the cell death potential of (ER stress inducer) thapsigargin. Very low doses of bortezomib (0.5-μM) and thapsigargin (0.5-μM) were enough to achieve very high cell death of dormant T24 cells in 96 hours. Single agent thapsigargin and bortezomib were noted to induce <10% cell death at 0.5-μM concentrations, however the combination induced approximately 90% cell death, significantly higher than their individual selves (p<0.0001, One way ANOVA) (FIG. 5D and S17A, SI). The combination index of the drug combination (thapsigargin and bortezomib) was calculated as 0.1±0.03 (Chou-Talalay method) indicating very strong synergy between the two drugs for ablation of the dormant cancer cell phenotype. Thapsigargin in concert with 0.5-μM wortmannin did not induce synergistic cell death (FIG. 5D). The combination of thapsigargin-bortezomib was much better than thapsigargin-wortmannin (FIG. 5D) in inducing dormant bladder cancer cell death. Live-dead staining using calcein AM and ethidium homodimer-1 after drug treatment yielded similar results as observed using the XTT assay.

The mode of action of thapsigargin has been shown to be calcium depletion in the ER leading to malfunctioning of multiple calcium dependent ER chaperones, causing misfolded of proteins and subsequent ER stress and unfolded protein response. It is likely that these misfolded proteins in ER are removed via the proteasome degradation pathway and not autophagy. This is the likely reason, the combination of ER stress and proteasome inhibition caused significant cell death. Inducing ER stress in concert with proteasome pathway likely caused chronic misfolded protein accumulation, leading to mitochondrial depolarization and cell death. Pro-apoptotic transcription factor CHOP was significantly upregulated in dormant cells treated with bortezomib and thapsigargin for 24 hours. We also observed that pan-caspase inhibitor (z-VAD-fmk) significantly rescued dormant spheroids from thapsigargin-bortezomib induced cell death (FIG. 5D) (p<0.0001, unpaired t-test) indicating a definite caspase involvement in the dormant cancer cell death during combination drug treatment. It is likely that pro-apoptotic proteins (Bax, Bak and CHOP) upregulated under chronic ER stress puncture mitochondrial outer-membrane and release apoptosis causing machinery (Smac-Diablo, Cytochrome C and pro-caspase 9) leading to a caspase dependent death (apoptosis). Bortezomib and thapsigargin have been shown to cause multiple side effects as single agents (41), which warrants the exploration of synergistic systems that could reduce the drug load and its associated side effects on patients whilst maintaining efficacy. Our study shows that these drugs act in synergy, thus requiring a much-reduced load of each drug, whilst maintaining efficacy.

### 2.7.1.1 Drugs Against Dormancy: Modulating Intracellular Calcium Levels to Achieve Accelerated Cell Death Under Chronic ER Stress

Although the combination of bortezomib-thapsigargin induced nearly complete death at very low concentrations of 0.5-μM each, it required approximately 96 hours to do so. Kinetic measurement of dormant cancer cell death revealed very low cell death (%) after 24 hours treatment with bortezomib and thapsigargin (<2% death) (FIG. 5E). Near total cell death (˜90%) was achieved only after 96 hours of drug exposure (FIG. 5E). We hypothesized that artificially raising calcium concentration in the cytoplasm could accelerate cell death under chronic ER stress conditions. It is known that the second response pathway under chronic ER stress involves efflux of calcium ions from the ER and cytoplasm into the mitochondrial matrix via the VDAC-IP3R-MCU set of pores, causing its depolarization. Increased calcium influx into the mitochondrial matrix erases the potential difference between two mitochondrial membranes causing mitochondrial depolarization, swelling, lysis and eventually cell death. Hence, we hypothesized that artificial elevation of intracellular cytoplasmic calcium concentration could lead to eventual higher total calcium in mitochondria. Artificial elevation of intracellular cytoplasmic calcium concentration could provide larger calcium pool, which under chronic ER stress could accelerate the erosion of mitochondrial potential difference leading to acceleration in apoptosis.

However, calcium influx into the cell is very tightly regulated to prevent sudden necrotic death. Free calcium cannot enter an epithelial cell via passive diffusion; due to the presence of voltage gated ion channels (TRP vanilloid family of ion channels) that prevent their free entry. In addition, intracellular calcium spurts are immediately buffered between the ER and mitochondria apart from cell extrusion, removing it from the cytoplasm. However, in presence of thapsigargin, it is likely that ER would not be able to participate in the buffering activity of the intracellular cytoplasmic calcium spurts. Thapsigargin blocks calcium entry into the ER lumen, which would leave only mitochondria to do the buffering activity if any. This would favorably increase the total calcium ion pool in cytoplasm for mitochondrial depolarization under chronic ER stress.

In our experiments, even very high concentrations of free calcium in the cell culture media (40 mM) had minimal impact on dormant cell viability even after 96 hours of exposure reinforcing that free calcium cannot diffuse through the membrane to cause toxicity. Hence, we used calcium ionophore calcimycin (A23157) to artificially deliver and increase intracellular calcium concentrations (FIG. 5F). As shown in Fig (FIG. 5F), calcimycin induced a concentration dependent cell death for the 24-hour time point. In addition, application of free calcium (5 mM) with calcimycin caused an increase in the cell death % after 24 hours. Pan-caspase inhibition did not rescue cells from calcimycin-induced death for the 24-hour time point (10-μM or 5-μM calcimycin, 5 mM calcium, 5-μM zVAD-fmk). It is likely that at higher concentrations of the calcimycin, the cell death is predominantly necrotic due to rapid ion influx.

We chose two concentrations of calcimycin, 0.5-μM and 5-μM with 5 mM calcium chloride for administration to the dormant cell system along with bortezomib-thapsigargin. At higher concentrations of the calcimycin (5-μM), the combination of calcimycin (5-μM), calcium (5-mM), bortezomib (0.5-μM) and thapsigargin (0.5-μM) caused a significant increase (p<0.0001, one way ANOVA, multiple comparisons) in the total cell death induced in 24 hours (˜60% death) compared to bortezomib-thapsigargin combination (<2% cell death, 24 hours) or calcium-calcimycin combination (˜45% cell death, 24 hours) (FIG. 5E). Although the calcium, calcimycin combination induced the majority of the cell death (necrotic), combining all the 4 drugs together showed a significant improvement over the individual drugs. While bortezomib and thapsigargin combination treatment induced ˜90% death over 96 hours, inclusion of calcimycin (5-μM) and calcium induced the same amount of death within 48 hours. However, it is likely that most of this death is necrotic and not apoptotic as it was irreversible via pan-caspase inhibition.

Ten-fold lower concentration of calcimycin (0.5-μM) however, induced entirely different results in combination with bortezomib-thapsigargin. The individual combination of calcium (5 mM)-calcimycin (0.5-μM) or bortezomib-thapsigargin (0.5-μM each) was not toxic to dormant cells (>15% at 48 hours), however their combination together caused increased apoptotic cell death (˜60% in 48 hours) (FIGS. 5G-H). In other words, we noticed a prominent acceleration of cell death when low concentration of calcimycin (0.5-μM) (in presence or absence of calcium (5-mM)) was supplemented with bortezomib-thapsigargin (0.5-μM each) (FIG. 5G). The acceleration in cell death was sustained all throughout the 96 hours (FIG. 5G). The acceleration was reversed by pan-caspase inhibition using zVAD-fink, indicating an acceleration of apoptotic cell death during calcimycin (0.5-μM) supplementation with bortezomib-thapsigargin (FIG. 5H). Cell culture media is supplemented with 1.8 mM calcium chloride, which could be likely responsible for the activity of calcimycin when added individually.

To effectively deliver calcium to sensitize dormant cells, calcium-loaded DPPC liposomes were freshly prepared for drug delivery. Calcium chloride loaded DPPC liposomes prepared by sonication at 50° C. followed by multiple freeze-thaw method showed a maximum loading capacity of 11±7 mM calcium chloride and had an average diameter of 115 nm and zeta potential of +25 mV. Calcium loaded DPPC liposomes prepared by Messersmith et al. (44) using multiple freeze-thaw method showed an approximate loading of 25 mM, similar to our loading capacity. Similar to calcium-calcimycin, delivering calcium loaded liposomes in combination with bortezomib and thapsigargin to the dormant cells also caused acceleration in the cell death. Even raising the intracellular cytoplasmic calcium concentration by mere 175-μM caused the acceleration in cell death as shown in (FIG. 5I, p<0.05, one way ANOVA). We observed ˜37% cell death in 48 hours by using calcium liposomes (175-μM) with bortezomib and thapsigargin, which increased to ˜52% at 72 hours, both of them being significantly higher than the bortezomib, thapsigargin alone (FIG. 5I). However, it is likely that calcium loading in the liposome could be a limiting factor in this approach and higher calcium loading can lead to better cell death percentages, comparable to calcimycin. We also imaged and quantified intracellular calcium increases using Fura-4-AM dye after 3-hour treatment with calcimycin, calcium and calcium liposomes. Intracellular calcium fluorescence was significantly higher in all treatment cases compared to DMSO control (p<0.05, One-way ANOVA), indicating an increased intracellular calcium concentration that preceded the acceleration of apoptosis during calcium supplementation.

In addition, we also believe that artificial elevation of intracellular calcium concentration can accelerate apoptotic cell death induced by multiple other chemotherapeutic drugs that target mitochondria for their activity for any disease under consideration such as Doxorubicin, Daunorubicin, Mitoxantrone, Docetaxel, Pixantrone, paclitaxel, etoposide, cyclosporamide, vincristine, netropsin, epirubicin, bortezomib, carfilzomib, mitomycin, idarubicin, bleomycin, brefeldin A, 17AAG, methotrexate, amonafide, Pancratistatin, Silver carbene, Naphthyridine derivative, 2,5-diaziridinyl-3-(hydroxymethyl)-6-methyl-1,4-benzoquinone, PMT7, Sodium selenite, Arsenic trioxide, SL017, 11β (CAS 865070-37-7), Aspirin, Ellipticine, Curcumin, Resveratrol, Berberine, Cerulenin, Ruthenium complexes, Gamitrinibs, Celastrol, Metformin, OSU-53, CPI-613, Tigecycline, Brilliant green, Betulinic acid, honokiol, arsenite trioxide, 3-bromopyruvate, Lonidamine, oblimersen, erastin, Gossypol, Obatoclax, Methyl jasmonate, Clotrimazole, Clodronate, Furanonaphthoquinones, Motexafin gadolinium, thapsigargin, α-Tocopheryl succinate (α-TOS), gemcetabine, cisplatin etc.

### 2.7.1.2 Drugs Against Dormancy: Mechanistic Understanding of the Accelerated Death Achieved by Calcium Modulation During Chronic ER Stress

In order to get a mechanistic understanding of the underlying behavior leading to cell death, ER stress marker CHOP expression and mitochondrial depolarization were studied using western blotting and fluorescence microscopy respectively. For the individual 5-μM calcimycin (higher concentration) and with bortezomib-thapsigargin (0.5-μM each) dosage, we observed prominent CHOP expression and mitochondrial depolarization. While CHOP protein expression after calcium (5 mM), calcimycin (5-μM) treatment was significantly lesser than bortezomib-thapsigargin treatment (FIG. 6A); the mitochondrial depolarization and cell death % were significantly higher after 24 hours of treatment (FIG. 6B). Rapid ion influx caused by high concentration of calcimycin likely induces mitochondrial depolarization, while bortezomib-thapsigargin combination induces the depolarization via CHOP expression. Minor expression of CHOP protein after treatment with calcimycin, could most likely be a stress response (FIG. 6A). Rapid cation (magnesium and calcium) influx associated with calcimycin has been shown to induce apoptosis or necrosis in a cell line and concentration dependent way (45, 46). We believe that the activity of bortezomib-thapsigargin is primarily CHOP induced mitochondrial depolarization and apoptosis (slower) (reversible by pan-caspase inhibition), whereas high concentration of calcimycin is rapid membrane depolarization induced necrosis (faster) (irreversible by pan-caspase inhibition) (FIG. 5D). Massive influx of ions associated with calcimycin can not only induce rapid mitochondrial depolarization leading to apoptosis, but also can cause of influx of water into cytoplasm leading to cell swelling, lysis and necrosis mediated death (47).

Phase contrast images showed prominent cell surface blebbing and puncturing after treatment with 5-μM calcimycin alone and its calcium combination for 24 hours, which was not observed in bortezomib-thapsigargin combination (an indication of necrosis). These results reinforce that at high concentrations of calcimycin (5-10 μM), there is significant necrotic cell death (pan-caspase inhibition did not impact cell death potential of calcimycin), along with significant mitochondrial depolarization. As bortezomib-thapsigargin combination also induces mitochondrial depolarization (CHOP dependent), it is plausible that these drug regimens in combination cause increased mitochondrial depolarization, which is likely, their combined mode of action.

At lower concentrations of calcimycin 0.5-μM (with calcium 5 mM) and bortezomib-thapsigargin (0.5-μM each), the cell death induced was <15% in 48 hours each. However, their combination caused prominent (yet reversible) increase in apoptotic cell death (˜65% in 48 hours). No CHOP protein expression was seen at lower concentrations of 0.5-μM calcimycin, however there was CHOP expression present during bortezomib-thapsigargin-calcimycin treatment (all 0.5-μM) similar to that of previous data. Mitochondrial depolarization quantified after 24 hours of treatment with bortezomib-thapsigargin-calcimycin (all 0.5-μM) and calcium (5 mM) showed a significant increase in depolarization compared to bortezomib-thapsigargin alone (FIG. 6C). These results reinforce our understanding of the previous results of significantly higher apoptotic cell death after inclusion of low concentrations of calcimycin (with 5 mM calcium) with ER stress inducer-proteasome inhibitor combination.

We believe we are the first to report the use of proteasome inhibitors in concert with ER stress inducers to sensitize dormant cancer cells for cell death. In addition, we also show for the first time that using calcium ions could significantly accelerate the apoptotic cell death in dormant cell systems and can be used in combination with these drug regimens. Bortezomib is already FDA approved for multiple myeloma and carfilzomib represents the next generation of FDA approved drugs for proteasome inhibition (48). Mipsagargin, a PSMA-targeted thapsigargin prodrug is in clinical trials against multiple tumor types etc (49). We believe our results with these drugs will have a quicker path to clinical translation against dormant tumors. We have shown and used high-throughput Amikagel system as a drug-screening platform for identification of, for example, lead drug candidates relevant to tumor specific phenotypes with direct clinic applications with FDA approved drugs.

### 2.7.2. Drugs Against Relapse and Micrometastases Formation

Maintaining cancer cells in a dormant state is considered as an alternative to avoid tumor metastases, although relapse from dormancy remains a major concern in this approach. We used the high-throughput Amikagel-dormant T24 3DTM system to investigate for lead drugs that although might not kill dormant cells, can prevent their relapse from dormancy. We defined the criteria of these drugs as those that may not ablate dormant tumors, but can effectively inhibit relapse and escape of cells from dormant tumors. As shown in FIG. 7A, we found that docetaxel chemotherapeutic treatment significantly reduces escape from dormancy, 7-day T24 3DTMs were treated with different doses of docetaxel for 96 hours on AM3 Amikagel. The 3DTMs were then transferred to the AM1 Amikagel, and cell escape from the parental 3DTM was tracked. It was noticed that docetaxel treatment significantly reduced the number of cells escaping the mother 3DTM (FIG. 7A). We studied the cell cycle distribution of docetaxel treated dormant T24 3DTMs, in order to investigate whether docetaxel mediated reduction in relapse is due to the ablation of cells in G2/M phase. Docetaxel treatment did not change the number of cells in G2/M phase of the cell cycle. However, an increase in the number of cells in the pre G0/G1 phase of cell cycle was noted. It is likely that docetaxel treatment ablated cells exiting the G2/M phase but did not hamper their entry. Kogashiwa et al. previously reported that treatment with docetaxel significantly reduced activity of Cdc42 and cell invasion in head and neck cancer cells. Cdc42 is a member of the Rho family of small GTPases, and promotes formation of actin rich filopodia and their extension prior to cell migration. Activity of Cdc42 protein was also shown to be a critical for migration of 22Rv1 cells by Zins et al. We believe that similar mechanism of Cdc42 inhibition could be active in docetaxel mediated prevention of dormancy relapse from dormant 3DTMs along with elimination of dividing cells. All concentrations of docetaxel (12.5 μM-100 μM) reduced the number of cells shed from dormant T24 3DTM. Microcolony formation was also drastically reduced in case of docetaxel-treated cells, while untreated cells continued to demonstrate microcolony formation (FIG. 7D-E). However, the escape of some cells from the mother 3DTM as well as the lack of response of the parent 3DTM, are indicative of the challenges in restricting tumors to a dormant state when microenvironment conditions change, i.e. change in adhesivity and/or mechanical properties as in case of transfer from AM3 to AM1 Amikagel.

We also tested the activity of ROCK inhibitor (Y-27632) in dormant cell relapse and migratory escape. Y-27632 is an inhibitor of ROCK (Rho-associated protein kinase), which is closely linked to RhoA, another member of Rho GTPases. It is known that proteins Cdc42-Rac-RhoA play key roles in regulating changes to cellular cytoskeleton for cell migration. While Cdc42 is involved in promoting actin-rich filopodia and membrane protrusions, RhoA is involved in production of acto-myosin bundles and generating contractile force via phosphorylation of myosin light chains (MLC). Inhibition of ROCK using Y-27632 has been shown to be effective in inhibiting cell migration and proliferation of metastatic MCF-7 breast cancer cells. ROCK inhibition also reduced breast cancer cell metastases to human bone cores in the study. Apart from above, ROCK is also associated with other cellular activities such as regulating cell division and migration under low cell-adhesion conditions. In a study by Yang et al., ROCK inhibition in dormant MCF-7 breast cancer cells using Y-27632 was shown to increase cell proliferation, migration, invasion (upregulation of Rac-1 and disintegration of cell junctions) and dissipation of cells. While ROCK inhibition reduced the invasion of metastatic MCF-7 cells, it activated the dormant MCF-7 cells into invasion.

Our results of Y-27632 treatment on dormant bladder cancer cells supported those of Yang et al. We found an increase in the number of cells escape and migrate out of dormant mother spheroid treated with 20-μM Y-27632 for 96 hours on AM3 gel and transferred to AM1 gel thereafter. Although, more cells were observed to leave the mother spheroid after treatment with ROCK inhibitor after transfer, we did not observe any changes in the cell cycle profile of dormant cells on AM3 gels after 96 hours of 20-μM Y-27632 treatment. We observed that ROCK inhibitor treatment only increases the number of cells leaving the spheroid on AM1 gel, but does not reverse the substrate induced cell cycle arrest. In Yang et al.'s experiment, the invasiveness of dormant MCF7 cells was seen to be significantly higher after treatment with ROCK inhibitor, due to the reduction in the E-cadherin expression, leading to loosening of cell-cell contacts, yielding a more motile cell phenotype. It is likely that a similar mechanism could be active in our system. In short, our results show that Docetaxel is a much better drug to inhibit cell migration out of dormant bladder cancer spheroids than ROCK inhibitor Y-27632.

Taken together, our results demonstrate the simplicity of the Amikagel platform for studying tumor dormancy and relapse. We show high resistance to conventional chemotherapeutics by the dormant systems. We put forth a unique regimen of drugs that are very effective in complete ablation of dormant cancer cells and significantly inhibit tumor relapse and escape. We believe our hydrogel platform sets the stage for high-throughput studies for the discovery of drugs that can reduce cancer dormancy, relapse and micrometastasis.

### Experimental Procedures

Materials

Amikacin hydrate (AH) (referred to as amikacin henceforth), docetaxel, wortmannin, chloroquine, propidium iodide, ribonuclease-A, poly(ethyleneglycol) diglycidyl ether (PEGDE), sodium orthovanadate and sodium fluoride were purchased from Sigma-Aldrich (St. Louis, Mo.), and used without further purification. Bortezomib was obtained from Selleck Chem (Houston, Tex.). Mitoxantrone was obtained from Ontaroio Chemicals (Guelph, ON). Thapsigargin and ROCK inhibitor Y-27632 dihydrochloride were obtained from Santa Cruz Biotech (Dallas, Tex.). Caclimycin ionophore was obtained from RPI Corp (Mount Prospect, Ill.). 16:0 PC (DPPC) 1,2-dipalmitoyl-sn-glycero-3-phosphocholine was obtained from Avanti Lipids (Alabaster, Ala.) to prepare the liposomes. Calcium chloride dihydrate was obtained EMD millipore (Billerica, Mass.). Calcein AM/ethidium homodimer Live/Dead stain®, JC-1 Dye-Mitochondrial Membrane Potential Probe, AlexaFluor 568-Phalloidin (actin-binding), 4′, 6-diamidino-2-phenylindole (DAPI) and Collagenase Type-2 were purchased from Life Technologies, (Carlsbad, Calif.). T24 and UMUC3 human bladder cancer cells were obtained from Professor Christina Voekel-Johnson at Medical University of South Carolina, Charleston, S.C. as part of an existing collaboration. These cell lines were verified for their authenticity through Bio-Synthesis Inc (Lewisville, Tex.). PC3-eGFP prostate cancer cells were obtained as a gift from Anne Cress' lab and Experimental Mouse Shared Service (EMSS)/Cancer Center Support Grant (CCSG), University of Arizona Cancer Center. MTT and XTT cell proliferation kit, prostate stromal cells (WPMY-1) and PC3 prostate carcinoma cells were purchased from American Type Culture Collection (ATCC) (Manassas, Va.) [26]. Bj5ta human foreskin fibroblasts were obtained from Center for Biosignatures Discovery Automation (CBDA), Biodesign Institute, ASU as part of an existing collaboration. NIH3T3 murine fibroblasts were obtained from Professor David Capco, School of Life Sciences, Arizona State University, Tempe, Ariz. as part of an existing collaboration. Cell culture media—RPMI media, DMEM with L-glutamine, Pen-Strep solution: 10000 units/mL penicillin and 10000 μg/mL streptomycin in 0.85% NaCl were purchased from Hyclone (Logan, Utah). Fetal bovine serum (FBS) was purchased from Atlanta Biologicals (Flowery Branch, Ga.). Cell culture-treated 24 and 96 well plates were purchased from Corning Life Sciences (Corning, N.Y.). RIPA buffer, Halt protease inhibitor cocktail (100×), Super Signal West Femto Maximum sensitivity substrate, Fluo-4 Direct™ Calcium Assay kit were obtained from ThermoScientific (Waltham, Mass.). Tris, glycine, SDS, Blotting-Grade Blocker, 2× Laemmli Sample Buffer, Mini Precast PROTEAN gels and precision plus protein standards (Dual color) were obtained from BioRad (Hercules, Calif.). Growth Factor reduced (Basement membrane) Matrigel matrix was obtained from Corning (Bedford, Mass.) and molecular biology grade agarose was obtained from Fisher Scientific (Pittsburgh, Pa.). Amersham Hybond P 0.45 PVDF membrane was purchased from GE Healthcare (Buckinghamshire, UK). CHOP D46F1 Rabbit mAb primary antibody, β-actin Rabbit Ab primary antibody and anti-rabbit IgG HRP-linked secondary antibodies were obtained from Cell Signalling (Boston, Mass.). Nanopure water was used in all preparations.

Rheological Measurements

Rheological measurements were carried out at 25° C. with an AR-G2 rheometer (TA Instruments) using parallel-plate configuration in the oscillatory mode. Amikagel samples prepared as described in section 3.1, were cut into discs of ˜1 mm thickness and ˜10 mm diameter. Cut Amikagel discs were loaded between the plates till the samples were in contact with the upper and lower plates (normal force applied <0.1N). Once in contact, a dynamic frequency sweep over an angular frequency range of 0.1-62 rad/sec was conducted at a fixed strain of 0.1%. Storage (G′) and loss (G″) moduli were experimentally determined as a function of applied frequency and absolute shear modulus (|G*|). The storage modulus (G′) gives information about material elastic properties and its mechanical stiffness, while loss modulus (G″) provides information about the viscous/liquid properties of the material. Absolute shear modulus representing the stiffness of the hydrogel was calculated as |G*|=(G′²+G″²)^{0.5}.

Preparation of Calcium Liposomes

16:0 PC (DPPC) 1,2-dipalmitoyl-sn-glycero-3-phosphocholine was dissolved in chloroform at a concentration of 20 mg/mL. 1 mL of the lipid solution in chloroform was dried under a constant stream of filtered air. The lipid crust was dessicated for 24 hours under vacuum to remove all traces of chloroform. The lipid crust was hydrated in 1 mL of 200 mM calcium chloride solution. The mixture was vortexed for 30 seconds followed by heating at 55° C. for 60 minutes with vortexing every 15 minutes. Next, the liposomes were sonication for 30 minutes in a bath sonicator at a temperature of 50° C. The loading percent was increased inside the vesicles using repeated freeze thaw method. Lipid-calcium chloride solution was heated to 55° C. (above the transition temperature of the DPPC) for one minute and rapidly cooled to −60° C. 10 cycles of repeated freeze thaw were performed. The unencapsulated calcium was removed via ion exchange using Amberlite IR-120+, H⁺ form activated by incubating the resin in 1M HCl for 24 hours prior to the exchange. After the exchange, the solvent around liposomes was brought to 10 mM HEPES buffer, pH 7.4, adjusted using 10M NaOH. Encapsulated calcium content was determined using the atomic absorption spectroscopy at the range of 1-5 ug/mL (detection range of the instrument). Size and the zeta potential of the liposomes were measured using Malvern Zetasizer Nano (Malvern Instruments, MA).

Generation of 3D Tumor Microenvironment Models (3DTMs)

Cell Culture

3DTMs were generated using single cell lines or co-cultures of cancer cells with stormal cells on AM3 amikagel (Table 1). Different cancer cell lines including, T24 bladder cancer, PC3 and PC3-eGFP prostate cancer and MDA-MB-231 breast cancer were employed in the generation of 3DTMs. Stromal cells including NIH3T3 murine fibroblasts, BJ-5ta human foreskin fibroblasts, WPMY-1 human prostate stromal cells were also used. T24, MDA-MB-231, WPMY-1 and Bj-5ta cells were propagated in DMEM supplemented with 10% (v/v) fetal bovine serum and 1% (v/v) penicillin and streptomycin (Pen-Strep solution: 10000 units/mL penicillin and 10000 μg/mL streptomycin in 0.85% NaCl). For NIH3T3 cell propagation, 1% (v/v) sodium pyruvate was added and fetal bovine serum was replaced with calf serum in the media. For all the remaining cell lines, RPMI media supplemented with 10% (v/v) fetal bovine serum and 1% (v/v) penicillin and streptomycin was used.

Generation of 3DTMs Using Amikagels

1 ml of amikagel AM1, AM2 and AM3 pre-gel solutions were filtered through a 0.20 μm filter and 40 μl of the filtrate was added to each well of a 96 well plate. The plates were sealed with paraffin tape (Parafilm, Menasha, Wis.) and incubated in an oven maintained at 40° C. for 7.5 hours. After gelation, the surfaces of Amikagels were washed with 150 μl of Nanopure® water for 12 hours, in order to remove traces of unreacted monomers. All 3DTM experiments were set up by liquid overlay culture of cells on top of Amikagel surface in a total volume of 150 OL media/well; either 100,000 cancer cells alone (single culture) or 50,000 stromal cells followed by 50,000 cancer cells (co-culture) were incubated, unless indicated otherwise in specific cases. After 48 hours of incubation, 50% of the media in the wells was replaced with fresh media i.e. DMEM/RPMI+10% (v/v) FBS+1% (v/v) Pen-Strep at regular intervals of 48 hours. Care was taken to withdraw and add the media slowly so as to not perturb 3DTM formation. Fresh media was added every 48 hours following cell plating. For 3DTM generation on 24 well plates, 400 μl of pre-gel volume was used instead of 40 μl. Different co-culture 3DTM systems are represented as fibroblast/stromal cells-epithelial cells (e.g. NIH3T3-T24, WPMY-1-T24) to accurately indicate the sequence of their addition. In most cases, 3DTMs were formed 5-7 days following culture on Amikagels, whereas 3DTMs containing WPMY-1 cells formed within 24 hours.

Scanning Electron Microscopy (SEM) of 3DTMs

Unless otherwise stated, all materials were purchased from Electron Microscopy Sciences (EMS; Hatfield, Pa.) and used when fresh. 3DTMs, approximately ˜1 mm in diameter, were manually manipulated with a fire-polished glass Pasteur pipet and washed three times using phosphate-buffered saline (PBS) in wells of a 96-well plate. 3DTMs were cytologically preserved with 0.1 M cacodylate buffer made fresh with 2% glutaraldehyde and 1% formaldehyde at room temperature for 1 hour. Specimens were washed 5 times with 0.1 M cacodylate buffer, and subsequently post-fixed in 1% OsO₄ for 2 hours at room temperature in the dark. After extensive washes in nanopure deionized (DI) water, the samples were dehydrated through a graded ethanol series, and dried through the critical point of CO₂ using Blazer CPD 020. Spheroids were immobilized on aluminum stubs and sputter coated with Pt—Au using a Technics sputter coater. Images were collected according to the conditions described in section 3.6.

Actin Staining

Unless otherwise stated, all reagents used for immunocytochemistry were purchased from Sigma Aldrich (St. Louis, Mo.). Different 3DTMs were fixed in 4% formaldehyde in 1×PBS for 12 hours before their transfer to 30% sucrose solution (w/v) for another 12 hours. Fixed spheroids were collected in Tissue Tek and flash frozen in liquid nitrogen. Following this, 3DTMs were cut into thin sections of ˜40 μm thickness using a cryotome maintained at ˜14° C. The cut sections were placed on a positively charged glass slide and dried at 37° C. to facilitate the attachment of the cryosection to the charged slide. Once dried, the 3DTM sections were thawed in 100 mM PBS with 2% formaldehyde (v/v) added to it. Cells were permeabilized with an intracellular buffer (ICB) (ICB contained the following ingredients: 100 mM KCl, 5 mM MgCl₂, and 20 mM HEPES (pH 6.8)) with 2% formaldehyde and 0.1% Triton X-100 for 10 minutes at room temperature in the dark. Cells were washed 3 times for 15 minutes per wash in ICB containing 1% bovine serum albumin (ICB-BSA) with gentle agitation. Fluorophore-conjugated actin-binding drug, Alexa Fluor 568 Phalloidin was used in this study to label filamentous actin. Phalloidin was diluted from the stock at a 1:200 μL dilution in antibody dilution buffer (ICB modified to contain 0.01% Tween-20 and 1% non-fat dry milk) and allowed to incubate on the sections overnight in a humidified chamber protected from light at room temperature. The sections were washed 3×15 minutes with ICB-BSA the following morning, and the nuclear probe 4′, 6-diamidino-2-phenylindole (DAPI) was challenged at 1:100 dilution in ICB for 15 minutes at room temperature. The sections were subsequently mounted in Vectashield (Vector Labs) and the cover slips were sealed at the edges with optically clear nail polish. All images were collected on a Leica SP5 laser scanning confocal microscope housed in the WM Keck Bioimaging Facility at ASU. The images shown represent maximum projection overlays with the z-axis set to scan at intervals of 0.4 μm. Images were adjusted linearly for contrast and brightness.

Hematoxylin & Eosin (H&E) Staining of 3DTMs

Spheroidal 3DTMs, approximately 1 mm in the longest dimension, were collected by manual manipulation of a fire-polished glass pipette and subjected to several washes with PBS. The specimens were subsequently fixed overnight in freshly prepared Bouin's fluid (75% saturated solution of picric acid, 5% glacial acetic acid in neutral-buffered formalin (pH 7.0) at 4° C. with gentle agitation. Fixed specimens were dehydrated through graded ethanol series and embedded in Paraplast+. Serial 10 μm thick sections were collected on glass slides and incubated at 42° C. overnight. Paraffin was removed with histology-grade toluene, and the slides were rehydrated through an ethanol series in Coplin jars, and washed for 5 minutes in Barnstead Nanopure filtered water (resistance of 18.2 MR). Basophilic elements (e.g. nuclei) of the cells were stained using Mayer's hematoxylin for 15 minutes at room temperature followed by 20 minutes in running tap water. The samples were counterstained with eosin for 5 minutes and incubated through increasing graded ethanol series. Slides were briefly transitioned from ethanol to toluene and incubated in 100% toluene for 2 minutes. Drops (approximately 15 μL) of Permount served to mount glass cover slips permanently. The slides were dried in a chemical hood overnight and imaged with a Nikon inverted microscope fitted with an Olympus DP26 color camera housed in the W.M. Keck Bioimaging Facility at ASU.

Cell Cycle Analyses

Following five days of incubation on AM3 Amikagels, 3DTMs of T24 cells with NIH3T3 murine fibroblast cells were harvested for cell cycle analysis. Four or five individual 3DTMs of T24 cells alone or UMUC3 cells alone were harvested on the 7 h day after seeding on Amikagels, collected in an eppendorf tube. 50 μl of 5 mg/ml collagenase was added to 3DTMs prepared using fibroblast helper cells for 30 minutes at 37° C. in order to facilitate their disassembly by gentle pipetteing. Single cell 3DTMs were disassembled using manual pipetting. Disassembled 3DTM cells were then centrifuged at 200 r.c.f. in order to collect the cell pellet. The pellet was resuspended in a solution of 1% v/v 1× Triton-X, 5% (v/v) fetal bovine serum (FBS), 50 μg/mL propidium iodide, and 0.006-0.01 units/mL ribonuclease A. After incubation for 30 minutes on ice, cells were analyzed for their cell cycle profiles using flow cytometry; the propidum iodide (PI) signal was detected using an excitation at 535 nm and emission at 617 nm. Voltages of the FL2-A, SSC and FSC channels were adjusted in order to obtain best representative peaks for alignment of 2n (diploidy—G0/G1) peak to 200 intensity units during flow cytometry. FL2A (FL2-Area) provides the information regarding the pulse area of the emitted fluorescence signal (total cell fluorescence) whereas SSC and FSC provide the information regarding the forward scatter and the side scatter light from the sample. FSC is a measure of diffracted light from the sample proportional to cell surface area or size and SSC is proportional to cell granularity or internal complexity.

Investigation of Chemotherapeutic Drug Efficacy on 3DTM Viability

Different concentrations (12.5, 25, 50 and 100 μM) of the anticancer drugs docetaxel, (10-40 μM) mitoxantrone, or (250-500 μM) thioTEPA were added to 3DTMs formed after 5-7 days of initial cell seeding on Amikagel. Drugs were added to 3DTMs of T24 cells co-cultured with NIH3T3 murine fibroblasts on day 5, but added to 3DTMs of T24 cells alone on day 7 due to the different times required for their formation After 96 hours of drug exposure, 3DTMs were disassembled using 50 μl of 5 mg/ml collagenase for 20 minutes to allow disassembly for co-culture spheroids, and cell viability was assessed by Live/Dead® staining followed by flow cytometry. 3DTM viability was also measured using MTT/XTT assay as described previously. Bortezomib, thapsigargin and wortmannin drugs were added at different concentrations (0.5, 1, 5, 10 and 20 μM) for 96 hours followed with cell viability using XTT assay. A calcium concentration of 5 mM was used with calcimycin ionophore of different concentrations (0.1, 0.5, 1, 5 and 10 μM) were used to induce cell death. Calcium liposomes were also added at a concentration of 175 μM to the dormant cells.

Fluorescence Microscopy and Flow Cytometry of 3DTMs

Live-Dead® Staining

Viability of 3DTMs was also determined using the Live-Dead® (Calcein AM-Ethidium homodimer) staining assay. Fresh serum free DMEM media, containing a final concentration of 1 μM of calcein AM and 2 μM of ethidium homodimer-1 (EthD-1), was added to the 3DTMs, and fluorescence imaging was carried out using a Zeiss fluorescence microscope after incubating the reagents for 20 minutes. Green fluorescence emission of calcein-AM inside the live cells was detected using 38 HE filter set (Excitation: 470/40; Emission: 525/50) and red fluorescence of nucleic acid bound-EthD-1 was detected using a 43 HE filter set (Excitation: 550/25; Emission: 605/70). The extent of red and green fluorescence were indicative of the extent of viable and dead cells, respectively, in the 3DTM.

To quantify the percent dead cells using flow cytometer following drug treatment, 3DTMs were disassembled using collagenase as described previously (whenever necessary). Disassembled cells were suspended in 1×PBS containing a final concentration of 1 μM of Calcein AM and 2 μM of Ethidium Homodimer-1 (EthD-1). After incubating for 20 minutes, fluorescence images were collected as described in previous sections. Cell viability was further quantified using flow cytometry on the same sample using BD FACSAria™ III. Red fluorescence of EthD-1 was detected using a 43 HE filter set (Excitation: 550/25; Emission: 605/70).

Mitochondrial Membrane Potential Detection

Mitochondrial membrane potential after treatment with bortezomib, thapsigargin, calcimycin and calcium was identified using JC-1 dye. 100,000 T24 cells seeded on AM3 gels for a week were treated with bortezomib (0.5 μM) and thapsigargin (0.5 μM), calcimycin (5 μM), calcium (5 mM) and their respective combinations for 24 hours. After 24 hours, the 3DTMs were collected in eppendorf tubes and washed with 1×PBS. They were resuspended in DMEM media supplemented with 100× dilution of the JC-1 dye and distributed into 96 well plates. After incubation for one hour at 37° C., the cells were imaged using 38 HE filter set (Excitation: 470/40; Emission: 525/50) and a 43 HE filter set (Excitation: 550/25; Emission: 605/70). Green fluorescence indicated presence of mitochondrial depolarization whereas red emission indicated intact mitochondria.

Mitochondrial fluorescence in cells were counted using image-J software. The fluorescence image was converted to an 8-bit image and the threshold was adjusted to separate group of fluorescence spots from each other. The image was converted to a binary image of black and white. The watershed tool was used to introduce one pixel distance between close fluorescence spots. Next, the fluorescence spots were counted using the “Analyze Particles” tool. Spots below the area of 10 pixel2 were ignored.

### Intracellular Calcium Imaging

Dormant 3DTMs grown on AM3 Amikagels were treated with calcimycin (0.5 and 5 μM) with calcium (5 mM) and calcium liposomes (175 μM) for 3 hours (3 dormant 3DTMs each). Following 3 hours, the 3DTMs were disassembled using rapid pipetting and cells were collected by centrifugation. The cells were washed with 1×PBS and resuspended in 50 μL cell culture media+50 μL Fluo-4 dye solution (prepared as per vendor's recommendation). After incubation for 60 minutes, the cells were imaged at excitation: 494 nm, emission: 516 nm. Green color fluorescence was used as an indicator of intracellular calcium concentration. Intracellular calcium fluorescence was quantified using imageJ.

Investigation of Drug Penetration into 3DTMs

Different concentrations of mitoxantrone, a fluorescent anticancer drug, were used to study drug penetration into NIH3T3-T24 3DTMs. NIH3T3-T24 3DTMs were incubated with different concentrations of mitoxantrone for 24 hours. The 3DTMs were then fixed and cryosectioned as described in section 3.7.4. Mitoxantrone fluorescence was detected by exciting the drug at 633 nm (He/Ne laser; Leica microscope) and collecting the emission signal using a 670-nm long pass filter. Image J software (NIH, Bethesda, Md.) was used to evaluate the cellular fluorescence of mitoxantrone. The raw fluorescence intensity of mitoxantrone was acquired using the software and corrected to account for background fluorescence.

Western Blotting

100,000 cells seeded on AM3 Amikagels were treated with bortezomib (0.5 μM) and Thapsigargin (0.5 μM), Calcimycin (5 μM), Calcium (5 mM) and their respective combinations for 24 hours, following which the cells were collected, washed with ice-cold 1×PBS, and lysed in 500 μL of RIPA buffer (25 mM Tris*HCl pH 7.6, 150 mM NaCl, 1% NP-40, 1% sodium deoxycholate, 0.1% SDS) supplemented with sodium orthovanadate (1 mM), sodium fluoride (1 mM), and 100 μL of 100× Halt protease inhibitor cocktail at 4° C. The lysates were then sonicated thrice for 10-15 seconds on ice and collected by centrifugation (16,000 rpm, 20 minutes, 4° C.). Supernatants containing the whole cell protein were stored at −20° C. for subsequent use.

BCA assay was used to quantify the total protein content of the cell lysates. Equivalent quantities of whole cell proteins (4 pg) were combined with 2× Laemmli buffer containing 5% β-mercaptoethanol, and were heat denatured for 5 minutes at 95° C. Equal amounts of protein (4 μg) were then loaded into to the wells of pre-cast gels and run for 35 minutes at 200 V in running buffer (25 mM Tris, 190 nM glycine, and 0.1% SDS, pH=8.3). The proteins were transferred to a methanol-activated Hybond PVDF membrane for 30 minutes at 20 V in the transfer buffer (25 mM Tris, 190 mM glycine, and 20% methanol, pH=8.3). The Hybond PVDF membrane was rinsed with 1×PBST and blocked with 3% blocking buffer 3× for 5 minutes each. The membrane was then incubated with the primary antibody for 12 hours at 4° C. (1:1000 CHOP D46F1 Rabbit mAb primary antibody) and (1:5000 j3-actin Rabbit Ab primary antibody). After three washes with 3% blocking buffer, the membrane was incubated in the secondary antibody (Anti-rabbit IgG HRP-linked Antibody) at 1:2000 dilution in the dark for 2.5 hours at 25° C. Following three washes with 1×PBST, the membrane was developed using Super Signal West Femto Maximum sensitivity substrate and viewed under chemiluminescence.

Area of the proteins actin and CHOP on the nitrocellulose membranes were measured using image-J software. The CHOP proteins from treatment samples were then normalized to their actin content and expressed which was further expressed as a percentage of the live control.

Impact of Amikagel Chemo-Mechanical Properties on Resistance and Dormancy of T24 3DTMs.

T24 3DTMs were first formed on AM3 Amikagels, and transferred to AM1 Amikagels on the seventh day following initial cell seeding, in order to investigate the role of chemo-mechanical properties of Amikagels on 3DTM fate, dormancy and escape from dormancy. Upon transfer, 3DTMs were monitored for cell spreading and motility on the gel for an additional 7 days. After 7 days, cell cycle analysis was carried out on the all 3DTMs as described in the previous sections.

Long-term experiments were also carried out where 3DTMs were continuously monitored for 15 days after their transfer from AM3 gel to AM1 gel. In order to study the effect of drug treatment, T24 3DTMs were first treated with 0-100 μM docetaxel for 96 hours after 7 days of initial seeding of cells on AM3 gel. After 96 hours of this drug treatment, the 3DTMs were transferred to AM1 gel to study how different chemo-mechanical properties influenced escape from dormancy. Similar studies were also performed using ROCK inhibitor Y-27632 (20 μM).

RNA Sequencing

30 wells of 15 days old 3D-DTM and reactivated cells on AM1 gel were collected and lysed to extract total cellular RNA using RNeasy mini kit as per the Qiagen protocol. Collected RNA were quantified using Nanodrop spectrophotometer and quality was assessed via the RIN number (RNA integrity number) obtained by using an Agilent 2100 Bioanalyzer. Samples with RIN score of 7 and above were selected for further cDNA synthesis.

IntegenX's automated Apollo 324 robotic preparation system was used to reverse transcribe RNA into cDNA and for DNA library preparation. 50 ng of total RNA was used to begin cDNA synthesis process via fully automated reverse transcription process. cDNA synthesis is performed using a SPIA (Single Primer Isothermal Amplification) kit co-developed by IntegenX and NuGEN. cDNA content was quantified using Nanodrop spectrophotometer.

Shearing is performed on a Covaris M220 system. After the DNA has been sheared to approximately 300 base pair fragments, the Nanodrop is used again to quantify the DNA in order to calculate the appropriate amount of DNA necessary for library construction. 500 ng of sheared cDNA was used for library construction involving repair of the sheared ends of cDNA. Indexing and adaptor ligation (complimentary to the oligos attached to the illumina flow cell) were performed using Kapa Biosystems library preparation kit. Bead clean up was performed in every step of library construction. Kapa HiFi Library Amplification kit from Kapa Biosystems was used for post library amplification. 10 cycles of post library enhancement were performed followed by clean up using AMPure beads from Agencourt Bioscience/Beckman Coulter. Post-amplified library was quantified by running the sample on Agilent 2100 Bioanalyzer to estimate the average fragment size within the quality control range (Expected fragment size—400 base pairs). After quality control check, the adaptors were quantified by quantitative PCR (qPCR) using the library quantification kit from Kapa Biosystems for use on the ABI 12K FLEX Real-Time PCR machine. Sample was diluted to match the acceptable cluster density range of Illumina (300 and 800 K/mm²). Next, denaturation and clustering are performed as per Illumina's preparation protocol. After appropriate sample clustering on the flow cell, the flow cells were loaded into the NextSeq Series Desktop sequencer (Illumina) for high output 1×75 run.

RNA-seq reads for each sample were quality checked using FastQC v0.10.1 and aligned to the human genome build 38 (GRCh38) primary assembly from Ensembl Release 83 Database (http://ftp.ensembl.org/pub/release-83/fasta/homo_sapiens/) using STAR v2.5.1b. Cufflinks v2.2.1 was used to report FPKM values (Fragments Per Kilobase of transcript per Million mapped reads) and read counts. Differential expression (DE) analysis was performed with EdgeR package from Bioconductor v3.2 in R 3.2.3. Multi-dimensional scaling (MSD) plot was drawn by plotMDS, in which distances correspond to leading log-fold-changes between samples. For each comparison (condition 1 vs condition2), genes with false discovery rate (FDR) <0.05 were considered significant and log 2-fold changes of expression between conditions (log FC) were reported.

Although the embodiments are described in considerable detail with reference to certain methods and materials, one skilled in the art will appreciate that the disclosure herein can be practiced by other than the described embodiments, which have been presented for purposes of illustration and not of limitation. Therefore, the scope of the appended claims should not be limited to the description of the embodiments contained herein.

Statistical Analyses

Averages have been expressed as mean±SD. The effectiveness of the drug combinations were quantified using the combination index (CI) by Chou-Talalay method. Two-tailed t-test with 95% CI was used analyze and compare the percent cell death data of individual drugs. One-way ANOVA has been used to study the differences between the effectiveness of multiple drugs and their combinations. Tukey's multiple comparisons test was used during multiple pairwise comparisons whereas Dunnett's multiple comparisons test was used while comparing multiple means to a single one (control). p<0.05 indicated significance in the analyses. All analyses were performed using the Prism GraphPad software. All experiments have been performed at least n=2 independent experiments with three replicates each unless specified.

### REFERENCES

All publications, including but not limited to patents and patent applications, cited below are herein
1. Persidis A (1999) Cancer multidrug resistance. Nature biotechnology 17(1):94-95.
2. Kottke T, et al. (2013) Detecting and targeting tumor relapse by its resistance to innate effectors at early recurrence. Nature medicine.
3. Piez D, et al. (2012) Cancer dormancy: a model of early dissemination and late cancer recurrence. Clinical Cancer Research 18(3):645-653.
4. Shiozawa Y, et al. (2010) GAS6/AXL axis regulates prostate cancer invasion, proliferation, and survival in the bone marrow niche. Neoplasia (New York, N.Y.) 12(2):116.
5. Aguirre-Ghiso J A (2007) Models, mechanisms and clinical evidence for cancer dormancy. Nature Reviews Cancer 7(11):834-846.
6. LaBarbera D V, Reid B G, & Yoo B H (2012) The multicellular tumor spheroid model for high-throughput cancer drug discovery. Expert Opinion on Drug Discovery 7(9):819-830.
7. Sainz B, TenCate V, & Uprichard S L (2009) Three-dimensional Huh7 cell culture system for the study of Hepatitis C virus infection. Virology Journal 6.
8. Cukierman E, Pankov R, & Yamada K M (2002) Cell interactions with three-dimensional matrices. Current Opinion in Cell Biology 14(5):633-639.
9. Derda R, et al. (2009) Paper-supported 3D cell culture for tissue-based bioassays. Proceedings of the National Academy of Sciences 106(44): 18457-18462.
10. Kunz-Schughart L A, Kreutz M, & Knuechel R (1998) Multicellular spheroids: a three-dimensional in vitro culture system to study tumour biology. International Journal of Experimental Pathology 79(1):1-23.
11. Skardal A, Sarker S F, Crabbe A, Nickerson C A, & Prestwich G D (2010) The generation of 3-D tissue models based on hyaluronan hydrogel-coated microcarriers within a rotating wall vessel bioreactor. Biomaterials 31(32):8426-8435.
12. Weirether F J, Walker J S, & Lincoln R E (1968) A precise method for replicating suspension cultures of mammalian cells. Applied microbiology 16(6):841-844.
13. Cheng G, Tse J, Jain R K, & Munn L L (2009) Micro-Environmental Mechanical Stress Controls Tumor Spheroid Size and Morphology by Suppressing Proliferation and Inducing Apoptosis in Cancer Cells. PLoS ONE 4(2):e4632.
14. Lin R-Z, Chou L-F, Chien C-C M, & Chang H-Y (2006) Dynamic analysis of hepatoma spheroid formation: roles of E-cadherin and j31-integrin. Cell and tissue research 324(3):411-422.
15. Gildea J J, Golden W L, Harding M A, & Theodorescu D (2000) Genetic and phenotypic changes associated with the acquisition of tumorigenicity in human bladder cancer. Genes, Chromosomes and Cancer 27(3):252-263.
16. Nishida T, Yasumoto K, Otori T, & Desaki J (1988) The network structure of corneal fibroblasts in the rat as revealed by scanning electron microscopy. Investigative Ophthalmology & Visual Science 29(12):1887-1890.
17. Beacham D A, Amatangelo M D, & Cukierman E (2001) Preparation of Extracellular Matrices Produced by Cultured and Primary Fibroblasts. Current Protocols in Cell Biology, (John Wiley & Sons, Inc.).
18. Whatcott C, Han H, Posner R G, & Von Hoff D D (2013) Tumor-stromal interactions in pancreatic cancer. Critical Reviews™ in Oncogenesis 18(1-2).
19. Kunz-Schughart L A, Freyer J P, Hofstaedter F, & Ebner R (2004) The Use of 3-D Cultures for High-Throughput Screening: The Multicellular Spheroid Model. Journal of Biomolecular Screening 9(4):273-285.
20. Zheng X, Wise S, & Cristini V (2005) Nonlinear simulation of tumor necrosis, neo-vascularization and tissue invasion via an adaptive finite-element/level-set method. Bulletin of mathematical biology 67(2):211-259.
21. Brown J M & Giaccia A J (1998) The Unique Physiology of Solid Tumors: Opportunities (and Problems) for Cancer Therapy. Cancer Research 58(7):1408-1416.
22. Du Y, et al. (2006) 3D hepatocyte monolayer on hybrid RGD/galactose substratum. Biomaterials 27(33):5669-5680.
23. Barkan D, et al. (2008) Inhibition of metastatic outgrowth from single dormant tumor cells by targeting the cytoskeleton. Cancer research 68(15):6241-6250.
24. Yong F, et al. (2014) A new tumour suppression mechanism by p27Kip1: EGFR down-regulation mediated by JNK/c-Jun pathway inhibition. Biochemical Journal 463(3):383-392.
25. Hsiao A Y-C (2011) 3D spheroid culture systems for metastatic prostate cancer dormancy studies and anti-cancer therapeutics development.
26. Wells A, Griffith L, Wells J Z, & Taylor D P (2013) The dormancy dilemma: quiescence versus balanced proliferation. Cancer research 73(13):3811-3816.
27. Makridakis M, et al. (2009) Chromosomal and proteome analysis of a new T24-based cell line model for aggressive bladder cancer. Proteomics 9(2):287-298.
28. Al-Sukhun S & Hussain M (2003) Current understanding of the biology of advanced bladder cancer. Cancer 97(S8):2064-2075.
29. Mialhe A, et al. (2000) Expression of E-, P-, N-cadherins and catenins in human bladder carcinoma cell lines. The Journal of urology 164(3):826-835.
30. Blagosklonny M V (2011) Cell cycle arrest is not senescence. Aging (Albany N.Y.) 3(2):94-101.
31. Grandhi T S P, et al. (2013) Sensitizing cancer cells to TRAIL-induced death by micellar delivery of mitoxantrone. Nanomedicine (0): 1-14.
32. Petrylak D P (1999) Chemotherapy for advanced hormone refractory prostate cancer. Urology 54(6A):30-35.
33. Miyamoto H, et al. (2004) Tumor-stroma interaction of human pancreatic cancer: acquired resistance to anticancer drugs and proliferation regulation is dependent on extracellular matrix proteins. Pancreas 28(1):38-44.
34. Wu H-C, et al. (2013) Tuning cell cycle of insect cells for enhanced protein production. Journal of biotechnology 168(1):55-61.
35. Lytton J, Westlin M, & Hanley M R (1991) Thapsigargin inhibits the sarcoplasmic or endoplasmic reticulum Ca-ATPase family of calcium pumps. Journal of Biological Chemistry 266(26): 17067-17071.
36. Ogata M, et al. (2006) Autophagy is activated for cell survival after endoplasmic reticulum stress. Molecular and cellular biology 26(24):9220-9231
37. Lee A-H, Iwakoshi N N, Anderson K C, & Glimcher L H (2003) Proteasome inhibitors disrupt the unfolded protein response in myeloma cells. Proceedings of the National Academy of Sciences 100(17):9946-9951.
38. Malhotra J D & Kaufman R J (2011) E R stress and its functional link to mitochondria: role in cell survival and death. Cold Spring Harbor perspectives in biology 3(9):a004424.
39. Deniaud A, et al. (2008) Endoplasmic reticulum stress induces calcium-dependent permeability transition, mitochondrial outer membrane permeabilization and apoptosis. Oncogene 27(3):285-299.
40. Green D R & Amarante-Mendes G P (1998) The point of no return: mitochondria, caspases, and the commitment to cell death. Apoptosis: Mechanisms and Role in Disease, (Springer), pp 45-61.
41. Ashley J D, et al. (2014) Liposomal bortezomib nanoparticles via boronic ester prodrug formulation for improved therapeutic efficacy in vivo. Journal of medicinal chemistry 57(12):5282-5292.
42. Benham C, Davis J, & Randall A (2002) Vanilloid and TRP channels: a family of lipid-gated cation channels. Neuropharmacology 42(7):873-888.
43. Berridge M J, Lipp P, & Bootman M D (2000) The versatility and universality of calcium signalling. Nature reviews Molecular cell biology 1(1): 11-21.
44. Messersmith P B, Vallabhaneni S, & Nguyen V (1998) Preparation of calcium-loaded liposomes and their use in calcium phosphate formation. Chemistry of materials 10(1): 109-116.
45. Gwag B, et al. (1999) Calcium ionophores can induce either apoptosis or necrosis in cultured cortical neurons. Neuroscience 90(4): 1339-1348.
46. Costa A K, Schieble T M, Heffel D F, & Trudell J R (1987) Toxicity of calcium lonophore A23187 in monolayers of hypoxic hepatocytes. Toxicology and applied pharmacology 87(1):43-47.
47. Lemaire M & Halperin M L (2009) Rapid tumor cell swelling and bursting: beware of collateral damage. Molecular therapy: the journal of the American Society of Gene Therapy 17(8):1310.
48. Meyerhoff A (1999) US Food and Drug Administration approval of AmBisome (liposomal amphotericin B) for treatment of visceral leishmaniasis. Clinical infectious diseases 28(1):42-48.
49. Doan N T Q, et al. (2015) Targeting thapsigargin towards tumors. Steroids 97:2-7.
50. Wang S-h & Lin S-Y (2013) Tumor dormancy: potential therapeutic target in tumor recurrence and metastasis prevention. Experimental Hematology & Oncology 2(1):29.
51. Kogashiwa Y, Sakurai H, Kimura T, & Kohno N (2010) Docetaxel suppresses invasiveness of head and neck cancer cells in vitro. Cancer science 101(6): 1382-1386.
52. Zins K, Lucas T, Reichl P, Abraham D, & Aharinejad S (2013) A Rac1/Cdc42 GTPase-Specific Small Molecule Inhibitor Suppresses Growth of Primary Human Prostate Cancer Xenografts and Prolongs Survival in Mice. PloS one 8(9):e74924.
53. Liu S, Goldstein R H, Scepansky E M, & Rosenblatt M (2009) Inhibition of rho-associated kinase signaling prevents breast cancer metastasis to human bone. Cancer research 69(22):8742-8751.
54. Croft D R & Olson M F (2006) The Rho GTPase effector ROCK regulates cyclin A, cyclin D1, and p27Kip1 levels by distinct mechanisms. Molecular and cellular biology 26(12):4612-4627.
55. Yang S, Kim H, & Aspenstrom P (2014) ROCK Inhibition Activates MCF-7 Cells. PLoS ONE 9(2):e88489.
56. Ranganathan A C, Adam A P, Zhang L, & Aguirre-Ghiso J A (2006) Tumor cell dormancy induced by p38SAPK and E R-stress signaling: an adaptive advantage for metastatic cells? Cancer biology & therapy 5(7):729-735.
57. Morin P J (2003) Drug resistance and the microenvironment: nature and nurture. Drug resistance updates 6(4): 169-172.
58. Sethi T, et al. (1999) Extracellular matrix proteins protect small cell lung cancer cells against apoptosis: a mechanism for small cell lung cancer growth and drug resistance in vivo. Nature medicine 5(6):662-668.
59. Borst P, Evers R, Kool M, & Wijnholds J (2000) A family of drug transporters: the multidrug resistance-associated proteins. Journal of the National Cancer Institute 92(16): 1295-1302.

What is claimed is:

1. A cross-linked hydrogel, comprising:
an aminoglycoside, wherein the aminoglycoside is selected from the group consisting of:
2-[(1R,2R,3S,4R,5R,6S)-3-(diaminomethylideneamino)-4-[(2R,3R,4R,5 S)-3-[(2S,3S,4S,5R,6S)-4,5-dihydroxy-6-(hydroxymethyl)-3-(methylamino)oxan-2-yl]oxy-4-formyl-4-hydroxy-5-methyloxolan-2-yl]oxy-2,5,6-trihydroxycyclohexyl]guanidine (herein after Streptomycin),
(2R,3S,4R,5R,6R)-5-amino-2-(aminomethyl)-6-[(1R,2R,3S,4R,6S)-4,6-diamino-2-[(2S,3R,4S,5R)-4-[(2R,3R,4R,5S,6S)-3-amino-6-(aminomethyl)-4,5-dihydroxyoxan-2-yl]oxy-3-hydroxy-5-(hydroxymethyl)oxolan-2-yl]oxy-3-hydroxycyclohexyl]oxyoxane-3,4-diol (herein after neomycin or neomycin b),
(2S,3S,4R,5R,6R)-5-amino-2-(aminomethyl)-6-[(2R,3S,4R,5 S)-5-[(1R,2R,3S,5R,6S)-3,5-diamino-2-[(2S,3R,4R,5S,6R)-3-amino-4,5-dihydroxy-6-(hydroxymethyl)oxan-2-yl]oxy-6-hydroxycyclohexyl]oxy-4-hydroxy-2-(hydroxymethyl)oxolan-3-yl]oxyoxane-3,4-diol (herein after paromomycin),
(2R,3S,4R,5R,6R)-5-amino-2-(aminomethyl)-6-[(1R,2R,3S,4R,6S)-4,6-diamino-2-[(2S,3R,4S,5R)-3,4-dihydroxy-5-(hydroxymethyl)oxolan-2-yl]oxy-3-hydroxycyclohexyl]oxyoxane-3,4-diol (herein after ribostamycin),
(2R,3S,4S,5R,6R)-2-(aminomethyl)-6-[(1R,2R,3S,4R,6S)-4,6-diamino-3-[(2S,3R,4S,5S,6R)-4-amino-3,5-dihydroxy-6-(hydroxymethyl)oxan-2-yl]oxy-2-hydroxycyclohexyl]oxyoxane-3,4,5-triol (herein after kanamycin),
(2S)-4-amino-N-[(1R,2S,3S,4R,5 S)-5-amino-2-[(2S,3R,4S,5S,6R)-4-amino-3,5-dihydroxy-6-(hydroxymethyl)oxan-2-yl]oxy-4-[(2R3R,4S,5S,6R)-6-(aminomethyl)-3,4,5-trihydroxyoxan-2-yl]oxy-3-hydroxycyclohexyl]-2-hydroxybutanamide (herein after amikacin),
(2S)-4-amino-N-[(1R,2S,3S,4R,5 S)-5-amino-4-[(2R,3R,6S)-3-amino-6-(aminomethyl)oxan-2-yl]oxy-2-[(2S,3R,4S,5S,6R)-4-amino-3,5-dihydroxy-6-(hydroxymethyl)oxan-2-yl]oxy-3-hydroxycyclohexyl]-2-hydroxybutanamide (herein after arbekacin),
(2R,3S,4R,5R,6R)-5-amino-2-(aminomethyl)-6-[(1S,2R,3R,4S,6R)-4,6-diamino-3-[(2S,3R,4S,5S,6R)-4-amino-3,5-dihydroxy-6-(hydroxymethyl)oxan-2-yl]oxy-2-hydroxycyclohexyl]oxyoxane-3,4-diol (herein after as bekanamycin),
(2S,3R,4S,5S,6R)-4-amino-2-[(1S,2S,3R,4S,6R)-4,6-diamino-3-[(2R,3R,6S)-3-amino-6-(aminomethyl)oxan-2-yl]oxy-2-hydroxycyclohexyl]oxy-6-(hydroxymethyl)oxane-3, 5-diol (herein after as dibekacin),
(2S,3R,4S,5S,6R)-4-amino-2-[(1S,2S,3R,4S,6R)-4,6-diamino-3-[(2R,3R,5S,6R)-3-amino-6-(aminomethyl)-5-hydroxyoxan-2-yl]oxy-2-hydroxycyclohexyl]oxy-6-(hydroxymethyl)oxane-3,5-diol (herein after as tobramycin),
(1R,3S,5R,8R,10R,11S,12S,13R,14S)-8,12,14-trihydroxy-5-methyl-11,13-bis(methylamino)-2,4,9-trioxatricyclo[8.4.0.0^{3,8}]tetradecan-7-one (herein after as spectinomycin),
4-[3-amino-2,6-dihydroxy-5-(methylamino)cyclohexyl]oxy-6′-(1-amino-2-hydroxyethyl)-6-(hydroxymethyl)spiro[4,6,7,7a-tetrahydro-3aH-[1,3]dioxolo[4,5-c]pyran-2,2′-oxane]-3′,4′,5′,7-tetrol (herein after as hygromycin b),
2-[4,6-diamino-3-[3-amino-6-[1-(methylamino)ethyl]oxan-2-yl]oxy-2-hydroxycyclohexyl]oxy-5-methyl-4-(methylamino)oxane-3,5-diol (herein after as gentamicin),
(2R,3R,4R,5R)-2-[(1S,2S,3R,4S,6R)-4-amino-3-[[(2S,3R)-3-amino-6-(aminomethyl)-3,4-dihydro-2H-pyran-2-yl]oxy]-6-(ethylamino)-2-hydroxycyclohexyl]oxy-5-methyl-4-(methylamino)oxane-3,5-diol (netilmicin),
(2R,3R,4R,5R)-2-[(1S,2S,3R,4S,6R)-4,6-diamino-3-[[(2S,3R)-3-amino-6-(aminomethyl)-3,4-dihydro-2H-pyran-2-yl]oxy]-2-hydroxycyclohexyl]oxy-5-methyl-4-(methylamino)oxane-3,5-diol (herein after as sisomicin),
(2S)-3-amino-N-[(1R,2S,3S,4R,5S)-5-amino-4-[(2R,3R,4S,5S,6R)-6-(aminomethyl)-3,4,5-trihydroxyoxan-2-yl]oxy-2-[(2R,3R,4R,5R)-3,5-dihydroxy-5-methyl-4-(methylamino)oxan-2-yl]oxy-3-hydroxycyclohexyl]-2-hydroxypropanamide (herein after as isepamicin),
(2R,3R,4R,5R)-2-[(1S,2S,3R,4S,6R)-4,6-diamino-3-[[(2S,3R)-3-amino-6-[(1 S)-1-aminoethyl]-3,4-dihydro-2H-pyran-2-yl]oxy]-2-hydroxycyclohexyl]oxy-5-methyl-4-(methylamino)oxane-3,5-diol (herein after as verdamicin),
2-amino-N-[(1S,2R,3R,4S,5S,6R)-4-amino-3-[(2R,3R,6S)-3-amino-6-[(1 S)-1-aminoethyl]oxan-2-yl]oxy-2,5-dihydroxy-6-methoxycyclohexyl]-N-methylacetamide (herein after as astromicin),
(2R,3R,4S,5S,6S)-2-[[(2R,3S,4R,4aR,6S,7R,8aS)-7-amino-6-[(1R,2R,3S,4R,6S)-4,6-diamino-2,3-dihydroxycyclohexyl]oxy-4-hydroxy-3-(methylamino)-2,3,4,4a,6,7,8,8a-octahydropyrano[3,2-b]pyran-2-yl]oxy]-5-amino-6-(hydroxymethyl)oxane-3,4-diol; sulfuric acid (herein after as apramycin) and/or salt or hydrate thereof; and
a polymeric compound having a structure, wherein the structure is selected from the group consisting of: 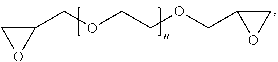
wherein n has a range of about 5 to about 135; 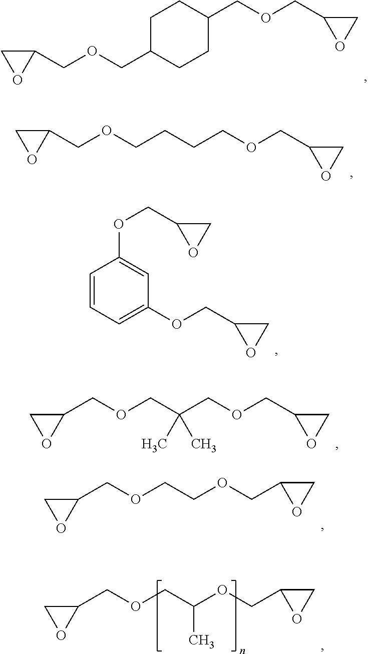
wherein n has a range of about 4 to about 15; and

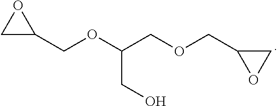 [SMILES: OCC(COCC1CO1)OCC1CO1]

2. The hydrogel of claim 1, wherein a mole ratio between said aminoglycoside and said polymeric compound is from about 1:1.5 to 1:3.

3. The hydrogel of claim 1, wherein said aminoglycoside is (2S)-4-amino-N-[(1R,2S,3S,4R,5 S)-5-amino-2-[(2S,3R,4S,5S,6R)-4-amino-3,5-dihydroxy-6-(hydroxymethyl)oxan-2-yl]oxy-4-[(2R,3R,4S,5S,6R)-6-(aminomethyl)-3,4,5-trihydroxyoxan-2-yl]oxy-3-hydroxycyclohexyl]-2-hydroxybutanamide, and/or salt or hydrate thereof.

4. The hydrogel of claim 1, wherein said polymeric compound having a structure of 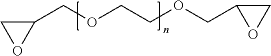
and n equals a first number such that an average molecular weight of the polymeric compound is about 500.

5. The hydrogel of claim 1, wherein said polymeric compound having a structure of 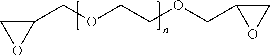
and n equals a second number such that an average molecular weight of the polymeric compound is about 2,000.

6. The hydrogel of claim 1, wherein said polymeric compound having a structure of 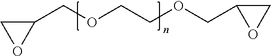
and n equals a third number such that an average molecular weight of the polymeric compound is about 6,000.

7. The hydrogel of claim 2, further comprising a mechanical stiffness of about 7 kilopascals (KPa) to about 100 KPa.

8. The hydrogel of claim 2, further comprising a non-adhesive surface.

9. A method to generate a 3D tumor microenvironment (3DTM) using the cross-linked hydrogel of claim 1, comprising overlaying a first plurality of cancer cells and culturing said cancer cells under conditions and for a duration sufficient to form a spheroidal 3DTM.

10. The method of claim 9, wherein the plurality of cancer cells comprises a seeding density of 1,000 to 50,000 cells.

11. The method of claim 9, wherein a size of the spheroidal 3DTM is dependent on the seeding density of the plurality of cancer cells.

12. The method of claim 9, wherein the plurality of cancer cells is selected from the group consisting of T24 bladder cancer cells, PC3 prostate cancer cells, PC3-eGFP prostate cancer cells, and MDA-MB-231 breast cancer cells.

13. The method of claim 9, wherein the spheroidal 3DTM comprises greater than 80% cells that are arrested in a G0/G1 phase of the cell cycle.

14. The method of claim 9, wherein the spheroidal 3DTM comprises greater than 95% cells that are arrested in a G0/G1 phase of the cell cycle.

15. The method of claim 9, wherein said culturing of said cancer cells results in the formation of dormant tumor cells in said spheroidal 3DTM.

16. The method of claim 9, wherein the overlaying further comprises a second plurality of stromal cells.

17. The method of claim 16, wherein the second plurality of stromal cells is selected from the group consisting of NIH3T3 murine fibroblasts, BJ-5ta human foreskin fibroblasts, and WPMY-1 human prostate stromal cells.

18. A method of screening an agent against tumor cells cultured in the spheroidal 3DTM of claim 9, comprising contacting said tumor cells with said agent and determining an effect on said cells.

19. The method of claim 18, wherein said agent is selected from the group consisting of a drug, antibody, and biologic.

20. The method of claim 19, wherein said agent is selected from the group consisting of Cabometyx (cabozantinib), Keytruda (pembrolizumab), Lenvima (lenvatinib), Opdivo (nivolumab), Sustol (granisetron), Syndros (dronabinol oral solution), Tecentriq (atezolizumab), Venclexta (venetoclax), Alecensa (alectinib), Cotellic (cobimetinib), Darzalex (daratumumab), Empliciti (elotuzumab), Farydak (panobinostat), Ibrance (palbociclib), Imlygic (talimogene laherparepvec), Keytruda (pembrolizumab), Lenvima (lenvatinib), Lonsurf (trifluridine and tipiracil), Ninlaro (ixazomib), Odomzo (sonidegib), Onivyde (irinotecan liposome injection), Opdivo (nivolumab), Opdivo (nivolumab), Portrazza (necitumumab), Tagrisso (osimertinib), Unituxin (dinutuximab), Varubi (rolapitant), Vistogard (uridine triacetate), Yondelis (trabectedin), Akynzeo (netupitant and palonosetron), Beleodaq (belinostat), Blincyto (blinatumomab), Cyramza (ramucirumab), Imbruvica (ibrutinib), Keytruda (pembrolizumab), Lynparza (olaparib), Opdivo (nivolumab), Zydelig (idelalisib), Zykadia (ceritinib), Gazyva (obinutuzumab), Gilotrif (afatinib), Imbruvica (ibrutinib), Kadcyla (ado-trastuzumab emtansine), Mekinist (trametinib), Pomalyst (pomalidomide), Revlimid (lenalidomide), Stivarga (regorafenib), Tafinlar (dabrafenib), Valchlor (mechlorethamine) gel, Xgeva (denosumab), Xofigo (radium Ra 223 dichloride), Abraxane (paclitaxel protein-bound particles for injectable suspension), Afinitor (everolimus), Afinitor (everolimus), Bosulif (bosutinib), Cometriq (cabozantinib), Erivedge (vismodegib), Iclusig (ponatinib), Inlyta (axitinib), Kyprolis (carfilzomib), Marqibo (vinCRIStine sulfate LIPOSOME injection), Neutroval (tbo-filgrastim), Perjeta (pertuzumab), Picato (ingenol mebutate) gel, Stivarga (regorafenib), Subsys (fentanyl sublingual spray), Synribo (omacetaxine mepesuccinate), Votrient (pazopanib), Xtandi (enzalutamide), Zaltrap (ziv-aflibercept), Abstral (fentanyl sublingual tablets), Adcetris (brentuximab vedotin), Afinitor (everolimus), Erwinaze (asparaginase Erwinia chrysanthemi), Lazanda (fentanyl citrate) nasal spray, Sutent (sunitinib malate), Sylatron (peginterferon alfa-2b), Vandetanib (vandetanib), Xalkori (crizotinib), Yervoy (ipilimumab), Zelboraf (vemurafenib), Zytiga (abiraterone acetate), Halaven (eribulin mesylate), Herceptin (trastuzumab), Jevtana (cabazitaxel), Provenge (sipuleucel-T), Xgeva (denosumab), Zuplenz (ondansetron oral soluble film), Afinitor (everolimus), Arzerra (ofatumumab), Avastin (bevacizumab), Cervarix [Human Papillomavirus Bivalent (Types 16 and 18) recombinant Vaccine] Elitek (rasburicase), Folotyn (pralatrexate injection), Istodax (romidepsin), Onsolis (fentanyl buccal), Votrient (pazopanib), Degarelix (degarelix for injection), Fusilev (levoleucovorin), Mozobil (plerixafor injection), Sancuso (granisetron), Treanda (bendamustine hydrochloride), Evista (raloxifene hydrochloride), Hycamtin (topotecan hydrochloride), Ixempra (ixabepilone), Tasigna (nilotinib hydrochloride monohydrate), Torisel (temsirolimus), Tykerb (lapatinib), Gardasil (quadrivalent human papillomavirus (types 6, 11, 16, 18) recombinant vaccine), Sprycel (dasatinib), Sutent (sunitinib), Vectibix (panitumumab), Arranon (nelarabine), Nexavar (sorafenib), Alimta (pemetrexed for injection), Avastin (bevacizumab), Clolar (clofarabine), Erbitux (cetuximab), Sensipar (cinacalcet), Tarceva (erlotinib, OSI 774), Aloxi (palonosetron), Bexxar, Emend (aprepitant), Iressa (gefitinib), Plenaxis (abarelix for injectable suspension), Premarin (conjugated estrogens), UroXatral (alfuzosin HCl extended-release tablets), Velcade (bortezomib), Eligard (leuprolide acetate), Eloxatin (oxaliplatin/5-fluorouracil/leucovorin), Faslodex (fulvestrant), Gleevec (imatinib mesylate), Neulasta, SecreFlo (secretin), Zevalin (ibritumomab tiuxetan) Zometa (zoledronic acid), Campath, Femara (letrozole), Gleevec (imatinib mesylate), Kytril (granisetron) solution, Trelstar LA (triptorelin pamoate), Xeloda, Zometa (zoledronic acid), Mylotarg (gemtuzumab ozogamicin), Trelstar Depot (triptorelin pamoate), Trisenox (arsenic trioxide), Viadur (leuprolide acetate implant), Aromasin Tablets, Busulflex, Doxil (doxorubicin HCl liposome injection), Ellence, Ethyol (amifostine), Temodar, UVADEX Sterile Solution, Zofran, Actiq, Anzemet, Camptosar, Gemzar (gemcitabine HCL), Herceptin, Inform HER-2/neu breast cancer test, Neupogen, Nolvadex, Photofrin, Proleukin, Sclerosol Intrapleural Aerosol, Valstar, Xeloda, Zofran, Anzemet, Bromfenac, Femara (letrozole), Gliadel Wafer (polifeprosan 20 with carmustine implant), Intron A (interferon alfa-2b, recombinant), Kytril (granisetron) tablets, Lupron Depot (leuprolide acetate for depot suspension), Miraluma test, Neumega, Quadramet (Samarium Sm 153 Lexidronam Injection), Rituxan, Taxol, Anexsia, Aredia (pamidronate disodium for injection), Arimidex (anastrozole), Campostar CEA-Scan, Elliotts B Solution (buffered intrathecal electrolyte/dextrose injection), Eulexin (flutamide), Feridex I.V., GastroMARK, Gemzar (gemcitabine HCL), Hycamtin (topotecan hydrochloride), Kadian, Leukine (sargramostim), Lupron Depot (leuprolide acetate for depot suspension), Photodynamic Therapy, Taxotere (Docetaxel), UltraJect, Visipaque (iodixanol), Zoladex (10.8 mg goserelin acetate implant), Ethyol (amifostine), Intron A (Interferon alfa-2b, recombinant), and Leukine (sargramostim).

21. The method of claim 18, wherein said contacting step comprises an agent that induces ER stress; and further comprises contacting said cells with a second agent that modulates intracellular calcium levels.
